(19) United States
(12) Patent Application Publication
(10) Pub. No.: US 2008/0319032 A1
(43) Pub. Date: Dec. 25, 2008

(54) TETRAHYDROBENZOTHIAZOLE ANALOGUES AS NEUROPROTECTIVE AGENTS

(76) Inventors: Nigel H Greig, Phoenix, MD (US); Mark Mattson, Bel Air, MD (US); Xiaoxiang Zhu, North Brunswick, NJ (US); Qian-Sheng Yu, Lutherville, MD (US); Harold Wayne Holloway, Middle River, MD (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/939,515

(22) Filed: Nov. 13, 2007

Related U.S. Application Data
(63) Continuation of application No. 10/332,115, filed on Sep. 15, 2003, now abandoned.
(60) Provisional application No. 60/216,388, filed on Jul. 6, 2000.

(51) Int. Cl. A61K 31/428; C12N 5/00; C07D 277/68; A61K 31/423; A61P 3/10; A61P 25/00; C07D 263/58

(52) U.S. Cl. 514/367; 435/375; 548/170; 548/221; 548/222; 514/375

(57) ABSTRACT

This invention relates generally to tetrahydrobenzothiazole analogues and tetrahydrobenzooxyzole analogues, to pharmaceutical compositions which include these compounds and a pharmaceutically acceptable carrier, and to methods of treatment using these compounds. The invention also encompasses pharmaceutically acceptable esters, amides, and salts of such compounds. The invention further provides a method of reducing or delaying apoptosis in a population of cells, comprising contacting the population of cells with a tetrahydrobenzothiazole analogue or a tetrahydrobenzooxyzole analogue, thereby reducing or delaying apoptosis in the population of cells.

### CROSS REFERENCE TO RELATED APPLICATIONS

This application is a Continuation of and claims the benefit of priority to U.S. patent application Ser. No. 10/332,115, filed Sep. 15, 2003, which application is a 35 U.S.C. § 371 National Phase entry of International Application Number PCT/US01/21504, filed Jul. 6, 2001, which application claims priority to U.S. Provisional Application Ser. No. 60/216,388, filed Jul. 6, 2000. The entire disclosures of the above-referenced priority application are incorporated by reference herein for all purposes.

### FIELD OF THE INVENTION

This invention relates generally to tetrahydrobenzothiazole analogues, to pharmaceutical compositions which include these compounds and a pharmaceutically acceptable carrier, and to methods of treatment using these compounds.

### BACKGROUND OF THE INVENTION

Deposition of neurotoxic forms of amyloid β-peptide (Aβ) in the brain likely contributes to neuronal degeneration and dementia in Alzheimer's Disease (AD) patients. The tumor suppressor protein p53 is a key modulator of stress responses, and activation of p53 precedes apoptosis in many cell types. Moreover, up-regulation of p53 has been described as a common feature of several neurodegenerative disorders including AD, Parkinson's Disease, stroke, trauma, brain or spinal cord injury, and excitoxic insults. Different triggers, like oxidative damage to DNA, overactivation of glutamate receptors, and disruption of cellular homeostasis, can initiate a cascade of intramolecular events that proceed via p53 activation of a death program called apoptosis. Consequently, the ability to inhibit p53 may be able to protect neurons against apoptotic insults.

In Komarov, P., et al., 285 Science 1733 (1999), pifithrin-α, a p53 inhibitor was studied for its efficiency in reducing the side effects of cancer therapy. This reference did not study novel analogues of tetrahydrobenzothiazoles. Tetrahydrobenzothiazole analogues have been synthesized in the prior art as antihelminthic compounds (anti-parasitic). One method, which is incorporated by reference in its entirety, is described in Singh, A., et al., 14B Indian J. Chem. 997 (1976), which references Saldabos, I., et al., I Khim. Farm. Zh. 27 (1967), 68 Chem. Abstr. 2856 (1968). In this method, tetrahydrobenzothiazole starting materials are treated with α-haloketones in a solvent.

### SUMMARY OF THE INVENTION

In accordance with the purpose(s) of this invention, as embodied and broadly described herein, this invention, in one aspect, relates to tetrahydrobenzothiazole analogues comprising one or more analogues of Formula (I):

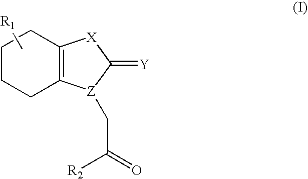

or a pharmaceutically acceptable salt or ester thereof, wherein:
X is O or S;
Y is NH, O, NR₂ or S;
Z is N or CH;

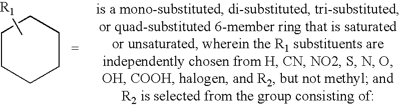

(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy, wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a second aspect, the invention relates to tetrahydrobenzothiazole analogues comprising one or more analogues of formula (II):

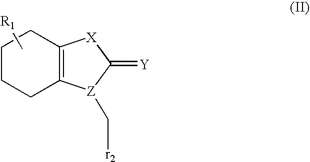

or a pharmaceutically acceptable salt or ester thereof, wherein:
X is O or S;
Y is NH, O, NR₂ or S;
Z is Nor CH;

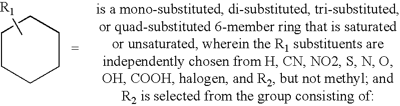

(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a third aspect, the invention relates to tetrahydrobenzothiazole analogues comprising one or more analogues of formula (III):

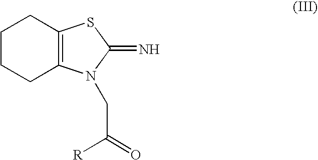

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy, wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl;
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a fourth aspect, the invention relates to tetrahydrobenzothiazole analogues comprising one or more analogues of formula (IV):

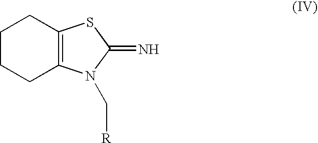

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a fifth aspect, the invention relates to tetrahydrobenzothiazole analogues comprising one or more analogues of formula (V):

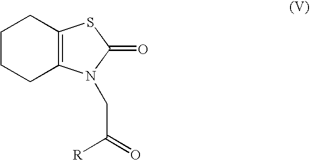

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a sixth aspect, the invention relates to tetrahydrobenzothiazole analogues comprising one or more analogues of formula (VI):

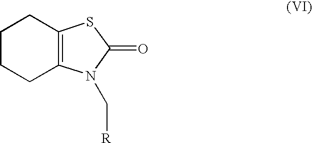

or a pharmaceutically acceptable salt or ester thereof, wherein:
R is selected from the group consisting of:
(a) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(b) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(c) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a seventh aspect, the invention relates to tetrahydrobenzothiazole analogues comprising one or more analogues of formula (VII):

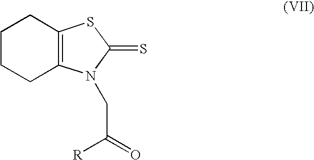

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In an eighth aspect, the invention relates to tetrahydrobenzothiazole analogues comprising one or more analogues of formula (VIII):

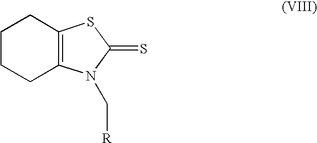

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a ninth aspect, the invention relates to tetrahydrobenzothiazole analogues comprising one or more analogues of formula (IX):

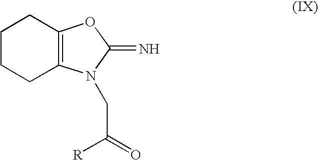

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

In a tenth aspect, the invention relates to tetrahydrobenzothiazole analogues comprising one or more analogues of formula (X):

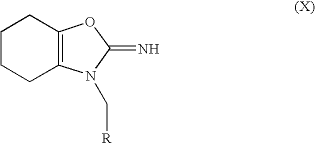

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

The invention also encompasses pharmaceutically acceptable esters, amides, and salts of such compounds, as will be explained in detail, infra.

Such compounds of the formula (I), (II), (III), (IV), (V), (VI), (VII), (VIII), (IX), (X), (XI) through (XV) and their pharmaceutically acceptable esters, amides, and salts are referred to herein as the inventive compounds.

In another aspect, the invention relates to pharmaceutical compositions containing the aforementioned inventive compounds in combination with a pharmaceutically acceptable carrier.

The invention further provides a method of reducing or delaying apoptosis in a population of cells, comprising contacting the population of cells with a tetrahydrobenzothiazole analogue, thereby reducing or delaying apoptosis in the population of cells.

In yet another aspect, the invention provides a method of treating a subject with a degenerative condition or of preventing a degenerative condition in a subject, comprising administering to the subject a therapeutically effective amount of a tetrahydrobenzothiazole analogue.

Furthermore, the invention provides a method of treating a subject after an ischemic event to reduce ischemia-induced apoptosis, comprising administering to the subject a therapeutically effective amount of a tetrahydrobenzothiazole analogue, thereby reducing apoptosis.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 provides the chemical structures and data for the compounds tested in examples 4 and 5.

FIG. 2 shows the effect PFTA (compound 5), its precursor, or analogues thereof on PC12 cells treated with the DNA-damaging agent camptothecin. PC12 cells were pretreated for 6 h with compounds (precursor, 1, 4-11, 13-15) prepared in 0.5% DMSO at concentrations between 100-400 nM) and were then exposed for 24 hours to camptothecin (40 μM). FIG. 2A shows the amount of fluorescence emitted by the live-cell indicator dye, 2′,7′-bis(2-carboxyethey)-5-6-carboxyfluorescein AM ester (BCECF AM: 5 μM). This fluorescent dye was taken up and retained by live cells. FIG. 2B shows the quantification of neuronal survival in the cultures (n=3 cultures). PFTα (compound 5) and several of its analogues protected cultured PC12 cells against death induced by camptothecin.

FIG. 3 shows the results of a neuronal survival assay using cultured hippocampal neurons treated with either camptothecin or etoposide in the presence and absence of PFTα (compound 5) or its precursor. PFTα protects cultured hippocampal neurons against death induced by DNA-damaging agents camptothecin and etoposide. Hippocampal cell cultures were pretreated for 1 hour with 0.5% DMSO (vehicle), 200 nM PFTα or 200 nM of PFTα precursor. Cultures were then exposed for 24 hours to the DNA-damaging agents camptothecin (5 μM) and etoposide (2.5 μM). Neuronal survival in each culture was quantified (n=4-6 cultures). **p<0.01 compared to corresponding value for cultures treated with vehicle or precursor (ANOVA with Scheffe post-hoc tests).

FIG. 4 shows the results of a neuronal survival assay using cultured hippocampal neurons treated with either camptothecin or etoposide in the presence and absence of PFTα and its analogues 14, 1, 15, 5x. Compounds were added 1 h before exposure of primary hippocampal neurons to the DNA-damaging compounds camptothecin (5 μM) or etoposide (2.5 μM). The percentage of neuronal survival 24 h after the treatment is given for each group as mean±SD.

FIG. 5 shows the results of a neuronal survival assay using cultured hippocampal neurons treated with camptothecin in the presence and absence of PFTα analogues 4, 6-8, 9-11, 13, 16 (100 nM). Compounds were added 1 h before exposure of primary hippocampal neurons to the DNA-damaging compound camptothecin (5 μM). Compounds 8 and 9 significantly protected against toxicity (p≦0.05).

FIG. 6 shows the results of histologic analysis of four different brain levels in mice following transient focal cerebral ischemia with and without pretreatment with PFT-α (compound 5). PFT-α (2 mg/kg) was administered intraperitoneally 1 h before transient middle cerebral artery occlusion in C57BL/6 mice. Twenty four hours after reperfusion, mice were euthanized and the infarct size was quantified after TTC-staining of 2 mm brain sections. FIG. 2A shows the infarct area in mm² at each level. FIG. 2B shows the infarct volume in mm³. Values are the mean and SD of 12 animals per group.

FIG. 7 shows the results of behavioral tests following PFT-α and compounds 5x and 13 treatment in a MPTP-induced Parkinson's disease model. C57BL/6 mice were given vehicle (control) or MPTP, and PFT-alpha (2 mg/kg), compound 5× (2 mg/kg), compound 13 (2 mg/kg), or vehicle were administered 30 minutes before the first MPTP injection and again 30 minutes after the last MPTP injection. Mice were tested on a rotarod apparatus. FIG. 7A shows the summary data for measurements of numbers of falls. FIG. 7B shows the summary data for running times. Values are the mean and SE (standard error) of determinations made in 12 mice/group. (*P<0.01 compared to control value, **P<0.01 compared to MPTP value. ANOVA with Scheffe post-hoc test).

FIG. 8 shows the results of densitometric analysis of Western blots stained with a TH antibody. PFT-alpha and compound 5x attenuate MPTP-induced loss of striatal tyrosine hydroxylase. Mice were given saline (control) or MPTP, and PFT-alpha (2 mg/kg), compound 5x (2 mg/kg), compound 13 (2 mg/kg) or vehicle were administered 30 minutes before the first MPTP injection and were repeated 30 minutes after the last MPTP injection. 7 days later, mice were euthanatized and striatal tissue samples were removed. Levels of TH were determined by immunoblot analysis (Western blots). The results of densitometric analysis of blots of samples from 4-6 different mice/group are shown as mean±SE. (*P<0.01 compared to control value, **P<0.01 compared to MPTP value. ANOVA with Scheffe post-hoc tests).

FIG. 9 shows the results of quantification of TH-positive cells in the substantia nigra of animals treated with MPTP, with or without PFT-α and compounds 5x and 13. PFT-α and compound 5x attenuated MPTP-induced loss of substantia nigra dopaminergic neurons. Mice were given saline (control) or MPTP, and PFT-alpha (2 mg/kg) (compound 5), Z-1-117 (2 mg/kg) (compound 5x), Z-1-143 (2 mg/kg) (compound 13) or vehicle were administered 30 minutes before the first MPTP injection and were repeated 30 minutes after the last MPTP injection. Values are shown the mean±SE of counts made in 4 mice/group. (*P<0.05, **P<0.01 compared to MPTP value. ANOVA with Scheffe post-hoc tests).

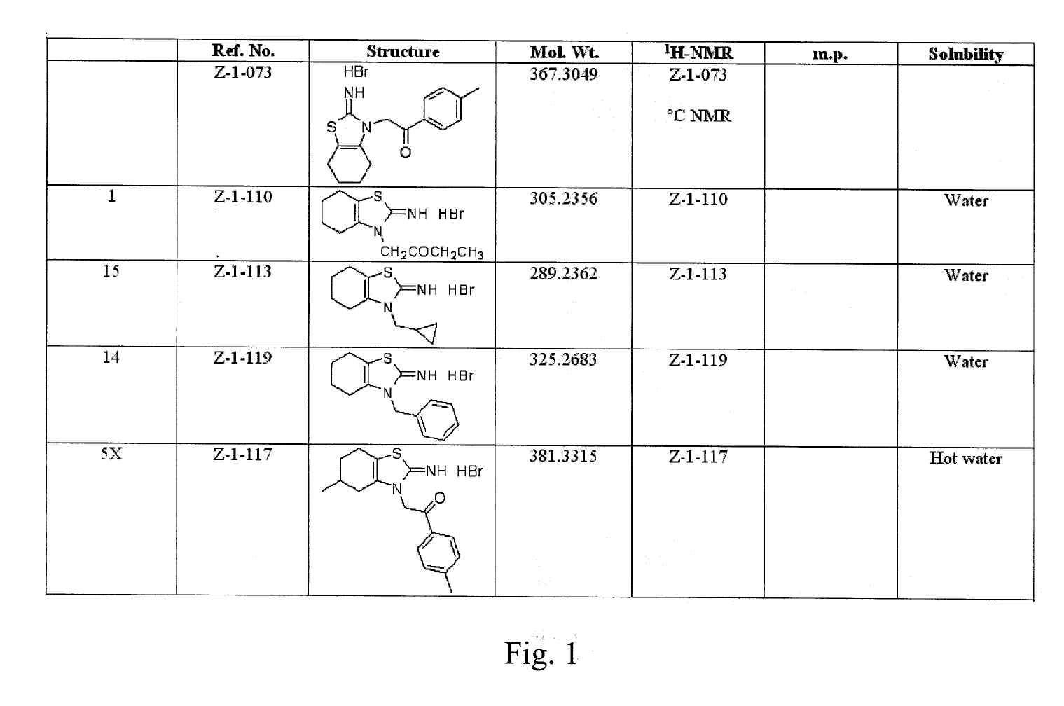

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods of using or making as such may, of course, vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “halogen” and “halo” refer to bromine, chlorine, fluorine, and iodine.

The term “alkyl” as used herein refers to a branched or unbranched saturated hydrocarbon group, such as methyl, ethyl, n-propyl, isopropyl, n-butyl, isobutyl, t-butyl, octyl, decyl, tetradecyl, hexadecyl, eicosyl, tetracosyl and the like. The term “lower alkyl” intends an alkyl group of from one to six carbon atoms, preferably from one to four carbon atoms. The term “cycloalkane” as used herein refers to a cyclic alkane group.

The term “alkoxy” as used herein intends an alkyl group bound through a single, terminal ether linkage; that is, an “alkoxy” group may be defined as —OR where R is alkyl as defined above. A “lower alkoxy” group intends an alkoxy group containing from one to six, more preferably from one to four, carbon atoms.

The term “alkylene” as used herein refers to a difunctional saturated branched or unbranched hydrocarbon chain, for example, methylene (—CH₂—), ethylene (—CH₂—CH₂—), propylene (—CH₂—CH₂—CH₂—), 2-methylpropylene[-CH₂—CH(CH₃)—CH₂—], hexylene [—(CH₂)₆—] and the like. “Lower alkylene” refers to an alkylene group of from 1 to 6, more preferably from 1 to 4, carbon atoms. The term “cycloalkylene” as used herein refers to a cyclic alkylene group.

The term “alkene” as used herein intends a mono-unsaturated or di-unsaturated hydrocarbon group. Asymmetric structures such as (AB)C═C(CD) are intended to include both the E and Z isomers. This may be presumed in structural formulae herein wherein an asymmetric alkene is present, or it may be explicitly indicated by the bond symbol —.

The term “aryl” as used herein refers to a C₆H₆ aromatic ring. Substituents on the aryl group may be present on any position, i.e. ortho, meta or para positions or fused to the aromatic ring.

By “bisphenol” it is meant that two C₆H₆ aromatic rings are present in a group in an unfused state. The aromatic rings may be joined at any position, i.e. ortho, meta or para positions, relative to the attachment position to the structure. Substituents on the bisphenol group may be present on or fused to any position of either aromatic ring.

The term “condensed aromatic” as used herein refers to more than one fused C₆H₆ aromatic ring. The aromatic rings may be fused at any bond i.e. along the C₂-C₃ bond relative to the C₁ attachment position to the structure. Substituents on the condensed aromatic group may be present on or fused to any position of any of the aromatic rings.

By “tetrahydrobenzothiazole” or “tetrahydrobenzothiazole analogue” it is meant to include tetrahydrobenzothiazole analogues, tetrahydrobenzooxyzole analogues, and heterocyclic analogues of the general formulas (GF1) and (GF2):

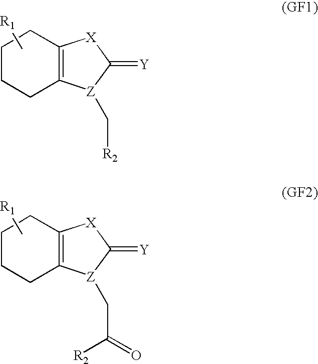

or a pharmaceutically acceptable salt or ester thereof, wherein:
X is O or S;
Y is NH, O, NR₂ or S;
Z is N or CH;

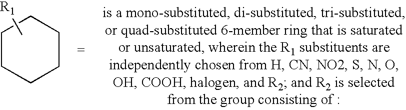

(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH and halogen;
(b) alkoxy which is straight chain, branched chain or cyclic, having a carbon chain length of 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

By the terms “PFTα,” “PFT-a,” and “pififthrin-α” is meant [2-(2-imino-4,5,6,7-tetrahydrobenzothiazol-3-yl)-1-p-tolyethanone] which is represented by the chemical structure (PFTα):

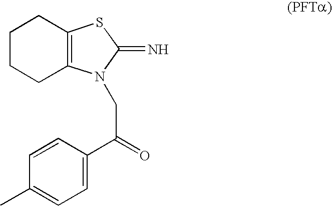

or a pharmaceutically acceptable salt or ester thereof.

By “substituted” is meant that the substituent group may be present anywhere within or attached to the substituted group.

By “neural cell” is meant any cell that can be located in the central or peripheral nervous system or is a precursor or derivative thereof, including, for example, neuronal cells, glial cells, neural stem cells, neuronal stem cells, neuroblasts. By “cardiac cell” is meant any cell that can be located in the cardiac tissue or is a precursor or derivative thereof, including, for example, cardiomyocytes, cardiac stem cells, endothelial cells, and myoblasts. By “pancreatic islet cell” is meant any cells type present in the pancreatic islet or precursors or derivatives thereof, including, for example, alpha cells (glucagon secreting cells) and beta cells (insulin secreting cells). By “muscle cells” is meant skeletal, smooth, or cardiac muscle cells or precursors or derivatives thereof, including, for example, myoblasts. Any of these cell populations can include immortalized cells and can include transfected or transformed cells. Thus, the population of neural cells, cardiac cells, pancreatic cells, or muscle cells can include one or more types of neural cells, cardiac cells, pancreatic cells, or muscle cells.

By “reducing or delaying” is meant either slowing or eliminating all or a portion of the apoptosis so that cell death is diminished or delayed in one or more cells in the cellular population.

As used throughout, by “contacting” is meant an instance of exposure of at least one cell (e.g., a neural cell, a stem cell, a cardiac cell) to an agent (e.g., a tetrahydrobenzothiazole analogue).

As used herein, “a degenerative condition” means a disease or condition marked by cell death. The degenerative condition can include, for example, neurodegenerative diseases (e.g., Alzheimer's disease, Parkinson's disease, Huntington's disease, amyotrophic lateral sclerosis, multiple sclerosis, brain and spinal cord injury, peripheral neuropathies, and stroke), degenerative cardiomyopathies (e.g., idiopathic dilated, ischemic, hypertrophic, obstructive, familial obstructive, familial arrhythmogenic right, ventricular, post-viral, alcoholic, endomyocardial fibrosis, amyloidosis, and muscular dystrophy), degenerative myopathies (e.g., muscular dystrophies), and other degenerative processes (e.g., diabetes Type I or Type II, and ischemia).

As used throughout, by “subject” is meant an individual. Preferably, the subject is a mammal such as a primate, and, more preferably, a human. Thus, the “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

“Optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyl where there is substitution.

In general, “a therapeutically effective dose or doses” means the amount needed to achieve the desired result or results (reducing or delaying apoptosis or treating a degenerative condition). One of ordinary skill in the art will recognize that the potency and, therefore, a “therapeutically effective dose or doses” can vary for the various tetrahydrobenzothiazole analogues used in this invention. One skilled in the art can readily assess the potency of the analogues.

The term “modified” is often used herein to describe polymers and means that a particular monomeric unit that would typically make up the pure polymer has been replaced by another monomeric unit that shares a common polymerization capacity with the replaced monomeric unit. Thus, for example, it is possible to substitute diol residues for glycol in poly(ethylene glycol), in which case the poly(ethylene glycol) will be “modified” with the diol. If the poly(ethylene glycol) is modified with a mole percentage of the diol, then such a mole percentage is based upon the total number of moles of glycol that would be present in the pure polymer but for the modification. Thus, in a poly(ethylene glycol) that has been modified by 50 mole % with a diol, the diol and glycol residues are present in equimolar amounts.

By “pharmaceutically acceptable” is meant a material that is not biologically or otherwise undesirable, i.e., the material may be administered to an individual along with the selected bicyclic compound without causing any undesirable biological effects or interacting in a deleterious manner with any of the other components of the pharmaceutical composition in which it is contained.

The invention also encompasses pharmaceutically acceptable nontoxic ester, amide, and salt derivatives of those compounds of formulas (I), (II), (III), (IV), (V), (VI), (VII), (VIII), (IX), and (X) containing a carboxylic acid moiety.

In one embodiment, the tetrahydrobenzothiazole analogue comprises one or more analogues of Formula (I):

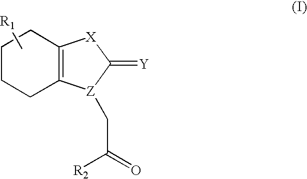

or a pharmaceutically acceptable salt or ester thereof, wherein:
X is O or S;
Y is NH, O, NR₂ or S;
Z is N or CH;

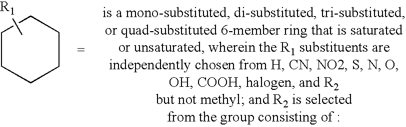

(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, preferably one or 3 to 8 carbon atoms, more preferably one, three or four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

In another embodiment, the tetrahydrobenzothiazole analogue comprises one or more analogues of formula (II):

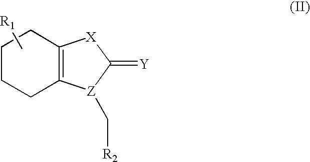

or a pharmaceutically acceptable salt or ester thereof, wherein:
X is O or S;
Y is NH, O, NR₂ or S;
Z is N or CH;

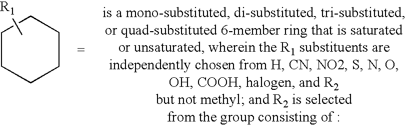

(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

In a further embodiment, the tetrahydrobenzothiazole analogue comprises one or more analogues of formula (III):

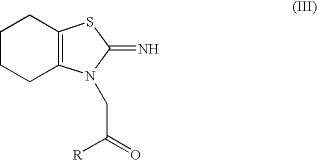

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, preferably one or 3 to 8 carbon atoms, more preferably one, three or four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Some preferred embodiments of formula (III) include where R is ethyl, methoxy, methoxyphenyl, fluorophenyl, chlorophenyl, nitrophenyl, or tetradecyloxyphenyl.

Moreover, in another embodiment, the tetrahydrobenzothiazole analogue comprises one or more analogues of formula (IV):

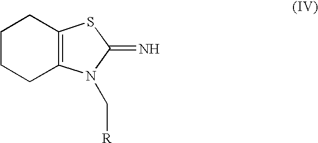

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (IV) include where R is ethyl, methoxy, methoxyphenyl, fluorophenyl, chlorophenyl, nitrophenyl, or tetradecyloxyphenyl.

In still a further embodiment, the tetrahydrobenzothiazole analogue comprises one or more analogues of formula (V):

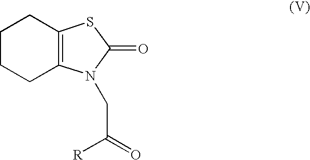

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₉ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (V) include where R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

In another preferred embodiment, the tetrahydrobenzothiazole analogue comprises one or more analogues of formula (VI):

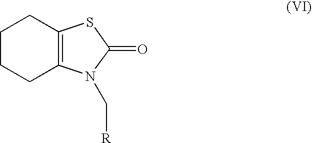

or a pharmaceutically acceptable salt or ester thereof, wherein:
R is selected from the group consisting of:
(a) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(b) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(c) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (VI) include where R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

In another preferred embodiment, the tetrahydrobenzothiazole analogue comprises one or more analogues of formula (VII):

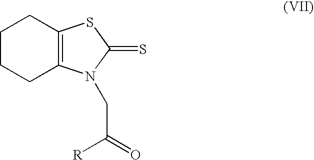

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (VII) include where R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

A preferred embodiment of the present invention includes the tetrahydrobenzothiazole analogue comprises one or more analogues of formula (VIII):

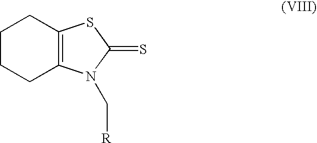

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₉ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (VIII) include where R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

In another preferred embodiment, the tetrahydrobenzothiazole analogue comprises one or more analogues of formula (IX):

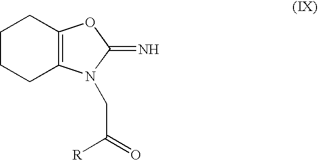

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (IX) include where R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

In another preferred embodiment, the tetrahydrobenzothiazole analogue comprises one or more analogues of formula (X):

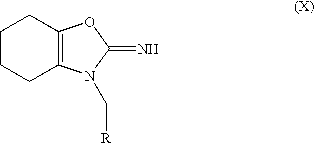

or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, preferably from 1 to 8 carbon atoms, more preferably from 1 to four carbon atoms, wherein the carbon chain is unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, preferably one to 8 carbon atoms, more preferably one to four carbon atoms, wherein the carbon chain is unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl; and
(e) condensed aromatic which is unsubstituted or substituted with at least one member selected from the group consisting CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; preferably substituted with at least one member selected from the group consisting of OH, COOH, halogen, and C₁-C₈ alkyl; more preferably substituted with at least one member selected from the group consisting of OH, COOH, Cl, F, Br, and C₁-C₄ alkyl.

Preferred embodiments of formula (X) include where R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

In a preferred embodiment for each of the tetrahydrobenzothiazole analogues having the formula (I), (II), (III), (IV), (V), (VII), (VIII), (IX), or (X), subparts (b), (c), (d), and (e) or formula (VI), subparts (a), (b) and (c) may preferably be C₁-C₈ alkenyl or C₁-C₈ alkynyl, more preferably C₁-C₄ alkenyl or C₁-C₄ alkynyl.

In one embodiment of the method of reducing or delaying apoptosis, the tetrahydrobenzothiazole analogue is pififthrin-α. Alternatively, the tetrahydrobenzothiazole analogue is selected from the group of analogues having formula (I), (II), (III), (IV), (V), (VI), (VII), (VIII), (IX), (X) or (XI) through (XV).

The present method is useful in reducing or delaying apoptosis induced by a toxin (e.g., the neurotoxic form of amyloid β-peptide, camptothecin, glutamate, anti-cancer drugs, such as etoposide and derivatives thereof; vinca alkaloids; and chemical agents, such as 3-nitropropionic acid, 1-methyl-4-phenyl-1,2,3,16-tetrahydropyridine (MPTP), domoic acid, and kainic acid), ischemia (e.g., caused by a stroke, transient ischemic attack, myocardial infarction), trauma (e.g., head injury), genetic defect (e.g., mutations in amyloid precursor protein; presenilins; α-synuclein; copper, zinc superoxide dismutase; Notch3 gene), genetic disease (e.g., muscular dystrophy, Huntington's disease, CADISIL (cerebral autosomal dominant arteriopathy with subcortical infarcts and leukoencephalopathy), environmental factors, (e.g., irradiation or severe seizure disorders).

Also provided is a method of treating a subject with a degenerative condition or of reducing one or more symptoms of a degenerative condition in a subject, comprising administering to the subject a therapeutically effective amount of a tetrahydrobenzothiazole analogue. The degenerative condition may result from events such as, but not limited to, surgery or organ transplant, trauma, severe seizure disorder, environmental factors, toxins, ischemia, genetic defects or diseases. With such conditions, one embodiment of the present invention provides a method of treating a subject before a surgical procedure to reduce apoptosis, comprising administering to the subject a therapeutically effective amount of a tetrahydrobenzothiazole analogue, thereby reducing apoptosis. In another embodiment, the invention provides a method of treating a subject after an excitoxic event. Such methods are also useful in treating a subject before a trigger event such as an ischemic or excitoxic event when such an event can be foreseen. In another embodiment, the invention provides a method of treating a subject after an ischemic event to reduce ischemia-induced apoptosis, comprising administering to the subject a therapeutically effective amount of a tetrahydrobenzothiazole analogue, thereby reducing apoptosis.

The invention also provides a method of treating a subject exposed to irradiation, comprising administering to the subject a therapeutically effective amount of a tetrahydrobenzothiazole analogue, thereby reducing one or more of the undesired symptoms of irradiation. In one embodiment, the irradiation is caused by radiation therapy. The irradiating event, in another embodiment, results in radiation poisoning. Thus, the analogue is administered before, during, or after the irradiating event.

Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. The reaction is conducted in water, alone or in combination with an inert, water-miscible organic solvent, at a temperature of from about 0° C. to about 100EC, preferably at room temperature. The molar ratio of compounds of structural formula (I) to base used are chosen to provide the ratio desired for any particular salts. For preparing, for example, the ammonium salts of the free acid starting material—a particular preferred embodiment—the starting material can be treated with approximately one equivalent of pharmaceutically acceptable base to yield a neutral salt. When calcium salts are prepared, approximately one-half a molar equivalent of base is used to yield a neutral salt, while for aluminum salts, approximately one-third a molar equivalent of base will be used.

Ester derivatives are typically prepared as precursors to the acid form of the compounds, as illustrated in the examples below, and accordingly may serve as prodrugs. Generally, these derivatives will be lower alkyl esters such as methyl, ethyl, and the like. Amide derivatives —(CO)NH₂, —(CO)NHR and —(CO)NR₂, where R is lower alkyl, may be prepared by reaction of the carboxylic acid-containing compound with ammonia or a substituted amine.

### Synthetic Methods:

The compounds of the invention may be readily synthesized using techniques generally known to synthetic organic chemists. Suitable experimental methods for making and derivatizing aromatic compounds are described, for example, in the references cited in the Background section herein above, the disclosures of which are hereby incorporated by reference for their general teachings and for their synthesis teachings. Methods for making specific and preferred compounds of the present invention are described in detail in the Examples.

### Utility and Administration:

The compounds of the invention defined by structural formulas (I), (II), (III), (IV), (V), (VI), (VII), (VIII), (IX), (X), (XI) through (XV), including the pharmacologically acceptable esters, amides or salts thereof, are useful in reducing or delaying apoptosis in a population of cells, comprising contacting the population of cells with a tetrahydrobenzothiazole analogue, thereby reducing or delaying apoptosis in the population of cells. In one embodiment, the cells are neural cells, and in another embodiment, the cells are cardiac cells. In another embodiment, the cells are pancreatic islet cells, and in yet another embodiment, the cells are muscle cells.

Preferably, the reduction or delay in apoptosis would be at least a 10% reduction or delay, including, for example, 15%, 20%, 25%, 30%, 40%, 50%, 60%, 70%, 80%, 90%, 100% or any amount in between. The reduction or delay can be measured, for example, by comparing the number of cells after contact with the tetrahydrobenzothiazole analogue to the number of cells in a control population of cells lacking contact with the tetrahydrobenzothiazole analogue. Histological signs of apoptosis that would be reduced or delayed in cells after contact with the tetrahydrobenzothiazole analogue include condensation of the chromatin, the occurrence of apoptotic bodies, and cellular shrinkage. DNA laddering and other signs of DNA degradation are also signs of apoptosis which would be reduced or delayed after contact with the tetrahydrobenzothiazole analogue. Apoptosis can also be assessed indirectly by observing, for example, a reduction in the amount of release or activity by the population of cells. Thus, if the cell population undergoes apoptosis, neurotransmitter release upon stimulation of neuronal cells would decrease. Similarly, a decrease in cardiac muscle contraction or cardiac output is an indicator of apoptosis. It is understood that one or a combination of indicators of apoptosis may show a delay or reduction.

The cell can be contacted in vitro with the agent, for example, by adding the agent to the culture medium (by continuous infusion, by bolus delivery, or by changing the medium to a medium that contains the agent) or by adding the agent to the extracellular fluid in vivo (by local delivery, systemic delivery, intravenous injection, bolus delivery, or continuous infusion). In vitro contact may be preferred, for example, in reducing or delaying apoptosis in a population of cells to be transplanted into a donor. In vivo contact may be preferred in reducing or delaying apoptosis in a subject with a disease, condition, or injury associated with apoptosis. The duration of “contact” with a cell or population of cells is determined by the time the agent is present at physiologically effective levels or at presumed physiologically effective levels in the medium or extracellular fluid bathing the cell or cells. Preferably, the duration of contact is 1-96 hours and, more preferably, for 24 hours, but such time would vary based on the half life of the agent and could be optimized by one skilled in the art using routine experimentation.

It is understood that the tetrahydrobenzothiazole analogue is administered in a therapeutically effective dose or doses to reduce or delay apoptosis. The agents used in this invention (i.e., tetrahydrobenzothiazole analogues) can be administered in vitro in an amount of about 10 nmol to 100 μmol. More preferably the agent is administered in vitro in an amount of about 1.0 mmol to 10 μmol. Necessary modifications in this dosage range may be determined by one of ordinary skill in the art.

The agents used in this invention are administered in vivo to a subject in need thereof by commonly employed methods for administering agents in such a way to bring the agent in contact with the population of cells. The agents of the present invention can be administered orally, parenterally, transdermally, extracorporeally, topically or the like, although intravenous administration is typically preferred.

The agents of the present invention can also be administered using gene therapy methods of delivery. See, e.g., U.S. Pat. No. 5,399,346, which is incorporated by reference herein. Using a gene therapy method of delivery, primary cells transfected with the gene for the agent of the present invention can additionally be transfected with tissue specific promoters to target specific organs, tissue, grafts, or cells.

The dosage of the agent varies depending on the type of disease or condition, degree of apoptosis, weight, age, sex, and method of administration. Also, the dosage of the agent varies depending on the target cell, tissue, graft, or organ. Generally, the agents can be orally or intravenously administered in vivo in an amount of about 0.01-1000 mg/kg. More preferably, the agent is administered in vivo in an amount of about 0.1 to 100 mg/kg. Even more preferably, the agent is administered in an amount of about 5 mg/kg. Thus, an administration regimen could include long-term, daily treatment. By “long-term” is meant at least two weeks and, preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed., latest edition), Mack Publishing Co., Easton, Pa. The dosage can also be adjusted by the individual physician in the event of any complication.

The agents can be administered conventionally as compositions containing the active agent as a predetermined quantity of active material calculated to produce the desired therapeutic effect in association with the required diluent, i.e., carrier or vehicle. Depending on the intended mode of administration, the agent can be in pharmaceutical compositions in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions of Formulas (I), (II), (III), (IV), (V), (VI), (VII), (VIII), (IX), (X) (XI) through (XV) include, as noted above, an effective amount of the selected compound in combination with a pharmaceutically acceptable carrier and, in addition, may include other medicinal agents, pharmaceutical agents, carriers, adjuvants, diluents, etc.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc. an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Thus, the compositions are administered in a manner compatible with the dosage formulation and in a therapeutically effective amount. As discussed above, precise amounts of active ingredient required to be administered depend on the judgment of the practitioner and are peculiar to each individual.

For oral administration, fine powders or granules may contain diluting, dispersing, and/or surface active agents, and may be presented in water or in a syrup, in capsules or sachets in the dry state, or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration, if used, is generally characterized by intradermal, subcutaneous, intramuscular, intraperitoneal, intravenous, intra-articular and intratracheal routes of injection. A more recently revised approach for parenteral administration involves use of a slow release or sustained release system such that a constant dosage is maintained. See, e.g., U.S. Pat. No. 3,610,795, which is incorporated by reference herein. The agents can also be administered using polymer based delivery systems, including, for example, microencapsulation as described in Langer (1998). Injectables can be prepared in conventional forms, either as liquid solutions or suspensions, solid forms suitable for solution or suspension in liquid prior to injection, or as emulsions.

For topical administration, liquids, suspension, lotions, creams, gels or the like may be used as long as the active compound can be delivered to the surface of the skin.

### Experimental

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in EC or is at ambient temperature, and pressure is at or near atmospheric.

The dotted lines attached to the benzene ring of structures 4-25 in the examples denote a chemical bond and not an additional carbon atom. Thus the R group of structure 4 is chemically bonded to Formula (XI) at the para position relative to the Fluorine atom. Throughout these examples, the dotted lines are meant to be a bond attachment and not an additional carbon atom.

Numbers in bold and parenthesis correspond to structures depicted in the examples for formula (XI) of example 1 and formula (XII) of example 2.

### Synthesis of the intermediate 2-amino-4,5,6,7-tetrahydrobenzothiazole

A mixture of cyclohexanone (1.96 g, 0.02 mmol), thiourea (3.04 g, 0.04 mmol) and iodine (5.08 g, 0.02 mmol) was stirred in a 110° C. oil bath for 12 h. The reaction mixture then was cooled, dissolved in boiling water and extracted with ether to remove any ketone and iodine. The solution was made basic with solid NaHCO₃, yellow crystals thereafter precipitated and were collected by filter to give 2-amino-4,5,6,7-tetrahydrobenzothiazole hydrogen iodide (3.2 g, 57%), m.p. 185-187° C. ¹H NMR (DMSO-d₆): 2.50-2.48 (m, 4H), 1.78-1.75 (m, 4H). ¹³C NMR (DMSO-d₆): 168.6, 140.7, 116.3, 26.1, 24.1, 24.0, 23.6. 2-amino-4,5,6,7-tetrahydrobenzothiazole hydrogen iodide (0.5 g) then was dissolved in hot saturated aqueous Na₂CO₃, and on cooling 2-amino-4,5,6,7-tetrahydrobenzothiazole was gained as white needle crystals (0.25 g, 93%), m.p. 87-88° C.

### Example 1

### Synthesis of Z-1-110: 1-(4,5,6,7-tertahydro-2-imino-3(2H)-benzothiazolyl)-2-butanone hydrobromide (1)

A mixture of 2-amino-4,5,6,7-tetrahydrobenzothiazole (76 mg, 0.49 mmol) and 1-bromo-2-butanone (51 μl, 0.50 mmol) in benzene (10 ml) was stirred at room temperature for two days. The resulting precipitate was filtered off from the reaction mixture and washed with a small amount of ethanol and benzene to give 1-(4,5,6,7-tertahydro-benzothiazolyl)-2-butanone hydrobromide (1) (75 mg, 50%) as pale yellow crystals. mp 114-115° C. ¹H NMR (DMSO-d₆): δ 9.45 (s, 1H), 5.06 (s, 2H), 2.64 (q, J=7.2 Hz, 2H), 2.41-2.52 (m, 2H), 2.29 (br, 2H), 1.73 (br, 4H), 0.99 (t, J=7.2 Hz, 3H); Anal. Calcd. HR-MS m/z calcd for C₁₁H₁₇N₂OS 225.1062, found 25.1071. Anal. Calcd. C₁₁H₁₇BrN₂OS: C, 43.18; H, 5.61. Found: C, 43.23; H, 5.56.

### Synthesis of PFT-α (Z-1-073): 1-(4-methylphenyl)-2-(4,5,6,7-tetrahydro-2-imino-3(2H)-benzothiazolyl)-ethanone hydrobromide (5)

A mixture of 2-amino-4,5,6,7-tetrahydrobenzothiazole (2) (154 mg, 1 mmol) and 2-bromo-4′-methylacetophone (213 mg, 1 mmol) in dry benzene (20 ml) was stirred at room temperature for two days. Thereafter, the precipitate was filtered off from the reaction mixture and washed with a small amount of benzene to afford 1-(4-methylphenyl)-2-(4,5,6,7-tetrahydro-2-imino-3(2H)-benzothiazolyl)-ethanone hydrobromide (5) (247 mg, 67%) as white crystals. mp 180° C. (EtOH/EtOAc) (lit²⁰: 182° C.); ¹H NMR (DMSO-d₆): δ 8.85 (s, 1H), 7.96 (d, J=8.1 Hz, 2H), 7.45 (d, J=8.1 Hz, 2H), 5.70 (s, 2H), 2.55-2.30 (m, 7H), 1.73 (m, 4H).

The following compounds were synthesized in accordance with the methods above, except the products were crystallized from MeOH/EtOAc, Z-1-189: ethyl 2-(4,5,6,7-tertahydro-2-imino-3(2H)-benzothiazolyl)-acetate hydrobromide (2): yield (52%); mp 224° C. (MeOH/EtOAc); ¹H NMR (DMSO-d₆) δ 9.63 (s, 1H), 4.93 (s, 2H), 4.21 (q, J=7.1 Hz, 2H), 2.38 (m, 2H), 1.73 (m, 4H), 1.24 (t, J=7.1 Hz, 3H),
ethyl 2-(4,5,6,7-tetrahydro-2-imino-3(2H)-benzothiazolyl)-acetate hydrohalide (2a),
methyl 2-(4,5,6,7-tetrahydro-2-imino-3(2H)-benzothiazolyl)-acetate hydrohalide (3),

Z-1-135: 1-(4-fluorophenyl)-2-(4,5,6,7-tertahydro-2-imino-3(2H)-benzothiazolyl)-ethanone hydrobromide (4): 44%, ¹H NMR (DMSO-d₆): 9.51 (s, 1H), 8.14 (d, J=5.5 Hz and J=8.7 Hz, 2H), 7.49 (t, J=8.7 Hz, 2H), 5.74 (s, 2H), 2.55-2.34 (m, 4H), 1.73 (m, 4H). HR-MS m/z calcd for C₁₅H₁₆FN₂OS 291.0967, found 291.0964. Anal. (C₁₅H₁₆BrFN₂OS) C, H, N,
1-(4-fluorophenyl)-2-(4,5,6,7-tetrahydro-2-imino-3(2H)-benzothiazolyl)-ethanone hydrohalide (4a),

Z-1-117: 1-(4-methylphenyl)-2-(4,5,6,7-tertahydro-2-imino-6-methyl-3(2H)-benzothiazolyl)-ethanone hydrobromide (5x): 54%; mp 254-256° C. (lit²⁰ 278° C.); ¹H NMR (DMSO-d₆): 9.60 (s, 1H), 7.95 (d, J=8.2 Hz, 2H), 7.45 (d, J=8.2 Hz, 2H), 5.70 (s, 2H), 2.71-2.64 (m, 1H), 2.51-2.31 (m, 5H), 2.21-2.12 (m, 1H), 1.83-1.79 (m, 2H), 1.36 (br, 1H), 1.02 (d, J=6.5 Hz, 3H). Anal. (C₁₇H₂₁BrN₂OS) C, H, N, 1-(4-methylphenyl)-2-(4,5,6,7-tetrahydro-2-imino-3(2H)-benzothiazolyl)-acetate hydrohalide (5xx),

Z-1-133: 1-(4-methoxylphenyl)-2-(4,5,6,7-tertahydro-2-imino-3(2H)-benzothiazolyl)-ethanone hydrobromide (6): 61%; ¹H NMR (DMSO-d₆): 9.47 (s, 1H), 8.03 (d, J=8.9 Hz, 2H), 7.16 (d, J=8.9 Hz, 2H), 5.67 (s, 2H), 3.89 (s, 3H), 2.55 (br, 2H), 2.32 (br, 2H), 1.73 (br, 4H). HR-MS m/z calcd for C₁₆H₁₉N₂O₂S 303.1167, found 303.1158. Anal. (C₁₆H₁₉BrN₂O₂S) C, H, N,
1-(4-methoxyphenyl)-2-(4,5,6,7-tetrahydro-2-imino-3(2H)-benzothiazolyl)-ethanone hydrohalide (6a),

Z-1-138: 1-(2-methoxylphenyl)-2-(4,5,6,7-tertahydro-2-imino-3(2H)-benzothiazolyl)-ethanone hydrobromide (7): 17%, ¹H NMR (DMSO-d₆): 7.70-7.12 (m, 4H), 5.42 (s, 2H), 3.99 (s, 3H), 2.50-2.39 (m, 4H), 1.81 (br, 4H). HR-MS m/z calcd for C₁₆H₁₉N₂O₂S 303.1167, found 303.1173. Anal. (C₁₆H₁₉BrN₂O₂S) C, H, N,
1-(2-methoxyphenyl)-2-(4,5,6,7-tetrahydro-2-imino-3(2H)-benzothiazolyl)-ethanone hydrohalide (7a),

Z-1-139: 1-(3-methoxylphenyl)-2-(4,5,6,7-tertahydro-2-imino-3(2H)-benzothiazolyl)-ethanone hydrobromide (8): 52%, mp 143-145° C.; ¹H NMR (DMSO-d₆): 9.50 (s, 1H), 7.67-7.37 (m, 4H), 5.74 (s, 2H), 3.86 (s, 3H), 2.55-2.33 (m, 4H), 1.73 (br, 4H). HR-MS m/z calcd for C₁₆H₁₉N₂O₂S 303.1167, found 303.1171. Anal. (C₁₆H₁₉BrN₂O₂S) C, H, N,
1-(3-methoxyphenyl)-2-(4,5,6,7-tetrahydro-2-imino-3(2H)-benzothiazolyl)-ethanone hydrohalide (8a),

Z-1-141: 1-(4-chlorophenyl)-2-(4,5,6,7-tertahydro-2-imino-3(2H)-benzothiazolyl)-ethanone hydrobromide (9): 70%; ¹H NMR (DMSO-d₆): 9.52 (s, 1H), 8.06 (d, J=8.6 Hz, 2H), 7.73 (d, J=8.6 Hz, 2H), 5.74 (s, 2H), 2.55-2.33 (m, 4H), 1.72 (br, 4H),
1-(4-chlorophenyl)-2-(4,5,6,7-tetrahydro-2-imino-3(2H)-benzothiazolyl)-acetate hydrohalide (9a),

Z-1-145: 1-(4-phenylphenyl)-2-(4,5,6,7-tertahydro-2-imino-3(2H)-benzothiazolyl)-ethanone hydrobromide (10): 58%; ¹H NMR (DMSO-d₆): 9.55 (s, 1H), 8.15 (d, J=8.5 Hz, 2H), 7.81 (d, J=7.0 Hz, 2H), 7.57-7.44 (m, 4H), 5.80 (s, 2H), 2.56-2.36 (m, 4H), 1.74 (br, 4H),
1-(4-phenylphenyl)-2-(4,5,6,7-tetrahydro-2-imino-3(2H)-benzothiazolyl)-acetate hydrohalide (10a),

Z-1-153: 1-(4-nitrophenyl)-2-(4,5,6,7-tertahydro-2-imino-3(2H)-benzothiazolyl)-ethanone hydrobromide (11): 61%, ¹H NMR (DMSO-d₆): 9.54 (s, 1H), 8.46 (d, J=8.9 Hz, 2H), 8.28 (d, J=8.9 Hz, 2H), 5.80 (s, 2H), 2.56-2.37 (m, 4H), 1.74 (br, 4H). HR-MS m/z calcd for C₁₅H₁₆N₃O₃S 318.0912, found 318.0903. Anal. (C₁₅H₁₆BrN₃O₃S) C, H, N,
1-(4-nitrophenyl)-2-(4,5,6,7-tetrahydro-2-imino-3(2H)-benzothiazolyl)-ethanone hydrohalide (11a),
1-(4-tetradecyloxyphenyl)-2-(4,5,6,7-tetrahydro-2-imino-3(2H)-benzothiazolyl)-ethanone hydrohalide (12),

Z-1-143: 1-phenyl-2-(4,5,6,7-tertahydro-2-imino-3(2H)-benzothiazolyl)-ethanone hydrobromide (13): 58%, mp 151-152° C.; ¹H NMR (DMSO-d₆): 9.53 (s, 1H), 8.07-8.04 (m, 2H), 7.79-7.74 (m, 1H), 7.66-7.62 (m, 2H), 5.75 (s, 2H), 2.55-2.33 (m, 4H), 1.72 (br, 4H). HR-MS m/z calcd for C₁₅H₁₇N₂OS 273.1162, found 273.1058. Anal. (C₁₅H₁₇BrN₂OS) C, H, N,
1 phenyl-2-(4,5,6,7-tetrahydro-2-imino-3(2H)-benzothiazolyl)-ethanone hydrohalide (13a), and

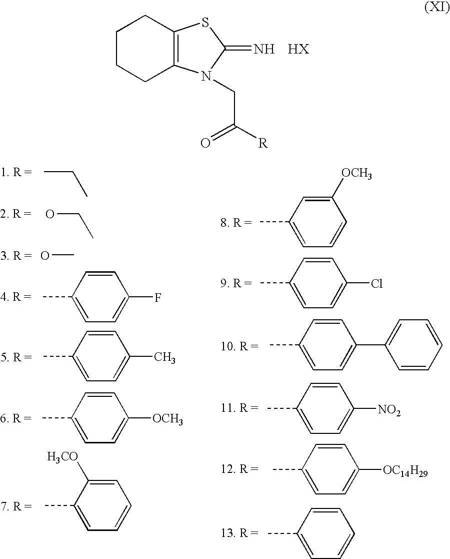

### Example 2

### Synthesis of Z-1-119: 3-(phenylmethyl)-4,5,6,7-tetrahydro-2(3H)-benzothiazolimine hydrobromide (14)

A mixture of 2-amino-4,5,6,7-tetrahydrobenzothiazole (101 mg, 0.66 mmol) and benzyl bromide (115 mg, 0.67 mmol) in THF (5 mL) was refluxed for two days. The precipitate was collected by filter, washed with a small amount of THF, and recrystallized from ethanol-ethyl ether to afford compound (14) (75 mg, 75%) as pale yellow crystals; mp 271° C.; ¹H NMR (DMSO-d₆): 9.63 (s, 1H), 7.45-7.35 (m, 3H), 7.15 (d, J=7.1 Hz, 2H), 5.29 (s, 2H), 2.50-2.30 (m, 4H), 1.76 (br, 4H); HR-MS m/z calcd for C₁₄H₁₇N₂S 245.1112, found 245.1104. Anal. (C₁₄H₁₇BrN₂S) C, H, N.
2-imino-3-benzyl-4,5,6,7-tetrahydro-3(2H)— benzothiazole hydrohalide (14a).

### Synthesis of Z-1-113: 3-(cyclopropylmethyl)-4,5,6,7-tetrahydro-2(3H)-benzothiazolimine hydrobromide (15)

A mixture of 2-amino-4,5,6,7-tetrahydrobenzothiazole (52 mg, 0.34 mmol) and (bromomethyl)cyclopropane (47 mg, 97%, 0.34 mmol) in ethanol (5 mL) was refluxed for one day. Thereafter, the solvent was evaporated under vacuum. The residue was extracted with hot benzene, filtered and washed with benzene to gain a pale yellow solid that then was recrystallized from ethanol-ethyl ether to afford (15) (60 mg, 61%) as pale yellow crystals; mp 239-240° C.; ¹H NMR (DMSO-d₆): 3.88 (d, J=7.0 Hz, 2H), 2.52-2.39 (m, 4H), 1.76-1.71 (m, 4H), 1.09 (br, 1H), 0.55-0.53 (m, 2H), 0.42-0.40 (m, 2H); HR-MS m/z calcd for C₁₁H₁₇N₂S 209.1112, found 209.1115. Anal. (C₁₁H₁₇BrN₂S) C, H, N.
2-imino-3-cyclopropylmethyl-4,5,6,7-tetrahydro-3(2H)-benzothiazole hydrohalide (15a),

The following compounds were synthesized in accordance with the methods for the synthesis of compounds (19) Z-1-165.

### Z-1-165: 2-imino-3-(4′-tert-butylbenzyl)-4,5,6,7-tetrahydro-3(2H)-benzothiazole hydrobromide (19)

A mixture of 2-amino-4,5,6,7-tetrahydrobenzothiazole (101 mg, 0.66 mmol) and 4-(tert-butyl)benzyl bromide (154 mg, 0.66 mmol) in THF (5 ml) was refluxed for two days. The resulting precipitate was collected by filter and washed with a small amount of THF and EtOH, and recrystallized from MeOH/EtOAc to give 2-imino-3-(4-tert-butylbenzyl)-4,5,6,7-tetrahydro-3(2H)-benzothiazole hydrobromide (98 mg, 39%) as white crystals.

mp 255-257° C. (MeOH/EtOAc); ¹H NMR (DMSO-d₆) δ 9.55 (s, 1H), 7.43 (d, J=8.4 Hz, 2H), 7.07 (d, J=8.4 Hz, 2H), 5.22 (s, 2H), 2.38 (m, 2H), 1.72 (m, 4H), 1.26 (s, 9H); Anal. (C₁₈H₂₅BrN₂S.0.25H₂O) C, H, N,
Z-1-167: 2-imino-3-phenylethyl-4,5,6,7-tetrahydro-3(2H)-benzothiazole (25),

The mixture of 2-amino-4,5,6,7-tetrahydrobenzothiazole (110 mg), 2-bromoethylbenzene (135 mg), KI (110 mg) in ethanol (5 mL) was refluxed for one day. The solvent was evaporated under vacuum. The residue was neutralized with saturated aqueous Na₂CO₃ solution. The product was separated from the aqueous solution as an uncrystallizable oil, and recrystallized from MeOH/EtOAc to give compound as pale brown crystal (yield 38%); mp 262° C. (MeOH/EtOAc); ¹H NMR (DMSO-d₆) δ 9.33 (br, 1H), 7.32-7.20 (m, 5H), 4.06 (t, J=6.9 Hz, 2H), 2.92 (t, J=6.9 Hz, 2H), 2.42 (m, 2H), 2.14 (m, 2H), 1.61 (m, 4H),
2-imino-3-phenylethyl-4,5,6,7-tetrahydro-3(2H)-benzothiazole hydrohalide (25a),

Z-1-181 2-imino-3-(4′-fluorobenzyl)-4,5,6,7-tetrahydro-3(2H)-benzothiazole hydrobromide (16), yield (41%); mp 252° C. (MeOH/EtOAc); ¹H NMR (DMSO-d₆) δ 9.61 (s, 1H), 7.30-7.23 (m, 4H), 5.26 (s, 2H), 2.36 (m, 2H), 1.71 (m, 4H),
2-imino-3-(4-fluorobenzyl)-4,5,6,7-tetrahydro-3(2H)-benzothiazole hydrohalide (16a),

Z-1-163: 2-imino-3-(2′,4′-difluorobenzyl)-4,5,6,7-tetrahydro-3(2H)-benzothiazole hydrobromide (17): yield (45%); mp 263-264° C. (MeOH/EtOAc); ¹H NMR (DMSO-d₆) δ 9.66 (s, 1H), 7.42-7.36 (m, 1H), 7.15-7.11 (m, 2H), 5.28 (s, 2H), 2.33 (m, 2H), 1.70 (m, 4H),
2-imino-3-(2,4-difluorobenzyl)-4,5,6,7-tetrahydro-3(2H)-benzothiazole hydrohalide (17a),

Z-1-179: 2-imino-3-(4′-methylbenzyl)-4,5,6,7-tetrahydro-3(2H)-benzothiazole hydrobromide (18): yield (39%); mp 253° C. (MeOH/EtOAc); ¹H NMR (DMSO-d₆) δ 9.58 (s, 1H), 7.23 (d, J=7.9 Hz, 2H), 7.05 (d, J=7.9 Hz, 2H), 5.22 (s, 2H), 2.35 (m, 2H), 2.29 (s, 3H), 1.70 (m, 4H),
2-imino-3-(4-methylbenzyl)-4,5,6,7-tetrahydro-3(2H)-benzothiazole hydrohalide (18a),

Z-1-161: 2-imino-3-(4′-nitrobenzyl)-4,5,6,7-tetrahydro-3 (2H)-benzothiazole hydrobromide (20): yield (44%); mp 235-236° C. (MeOH/EtOAc); ¹H NMR (DMSO-d₆) δ 9.75 (s, 1H), 8.37 (d, J=8.8 Hz, 2H), 7.51 (d, J=8.8 Hz, 2H), 5.52 (s, 2H), 2.59 (m, 2H), 2.41 (m, 2H), 1.80 (m, 4H),
2-imino-3-(4-nitrobenzyl)-4,5,6,7-tetrahydro-3(2H)-benzothiazole hydrohalide (20a),

Z-1-183: 2-imino-3-(4′-cyanobenzyl)-4,5,6,7-tetrahydro-3(2H)-benzothiazole hydrobromide (21): yield (32%); mp 247° C. (MeOH/EtOAc); ¹H NMR (DMSO-d₆) δ 9.64 (s, 1H), 7.94 (d, J=8.3 Hz, 2H), 7.35 (d, J=8.3 Hz, 2H), 5.38 (s, 2H), 2.33 (m, 2H), 1.73 (m, 4H),
2-imino-3-(4-cynobenzyl)-4,5,6,7-tetrahydro-3 (2H)-benzothiazole hydrohalide (21a),

Z-1-175: 2-imino-3-(4′-trifluorobenzyl)-4,5,6,7-tetrahydro-3(2H)-benzothiazole hydrobromide (22): yield (40%); mp 265-266° C. (MeOH/EtOAc); ¹H NMR (DMSO-d₆) δ 9.68 (s, 1H), 7.80 (d, J=8.0 Hz, 2H), 7.38 (d, J=8.0 Hz, 2H), 5.41 (s, 2H), 2.55 (m, 2H), 2.35 (m, 2H), 1.72 (m, 4H); Anal. (C₁₅H₁₆BrF₃N₂S.H₂O) C, H, N,
2-imino-3-(4-trifluoromethylbenzyl)-4,5,6,7-tetrahydro-3(2H)-benzothiazole hydrohalide (22a),
2-imino-3-(4-phenylbenzyl)-4,5,6,7-tetrahydro-3(2H)-benzothiazole hydrohalide (23),

Z-1-171: 2-imino-3-(naphthalenylmethyl-4,5,6,7-tetrahydro-3(2H)-benzothiazole hydrobromide (24): yield (34%); mp 268° C. (MeOH/EtOAc); ¹H NMR (DMSO-d₆) δ 9.66 (s, 1H), 8.0-7.92 (m, 3H), 7.64 (s, 1H), 7.56-7.53 (m, 2H), 7.33 (d, J=7.2 Hz, 1H), 5.44 (s, 1H), 2.54 (m, 2H), 2.39 (m, 2H), 1.68 (m, 4H).
2-imino-3-(2-menaphthyl)-4,5,6,7-tetrahydro-3(2H)-benzothiazole hydrohalide (24a),

Z-1-187: 2-Imino-3-butyl-4,5,6,7-tetrahydro-3(2H)-benzothiazole hydrobromide (26), yield (19%); ¹H NMR (DMSO-d₆) δ 9.34 (s, 1H), 3.87 (t, J=7.1 Hz, 2H), 1.77 (m, 4H), 1.57 (m, 2H), 1.34 (m, 2H), 0.91 (t, J=7.1 Hz, 3H),
2-imino-3-butyl-4,5,6,7-tetrahydro-3(2H)-benzothiazole hydrohalide (26a),

Z-2-007: 1-(4-methylphenyl)-2-(4,5,6,7-tetrahydro-2-imino-3(2H)-benzoxazolyl)-ethanone hydrobromide (27): yield (51%); mp 237° C. (MeOH/EtOAc); ¹H NMR (DMSO-d₆) δ 9.64 (br, 1H), 7.95 (d, J=8.0 Hz, 2H), 7.46 (d, J=8.9 Hz, 2H), 5.78 (s, 2H), 2.52 (br, 2H), 2.44 (s, 3H), 2.33 (m, 2H), 1.77 (m, 4H

Z-2-013: 1-(4,5,6,7-tertahydro-2-imino-3(2H)-benzoxazolyl)-2-butanone hydrobromide (28): yield (57%); mp 206° C. (MeOH/EtOAc); ¹H NMR (DMSO-d₆) δ 9.56 (s, 1H), 4.95 (s, 2H), 2.59 (q, J=7.2 Hz, 2H), 2.27 (m, 2H), 1.78-1.69 (m, 4H), 0.99 (t, J=7.2 Hz, 3H),

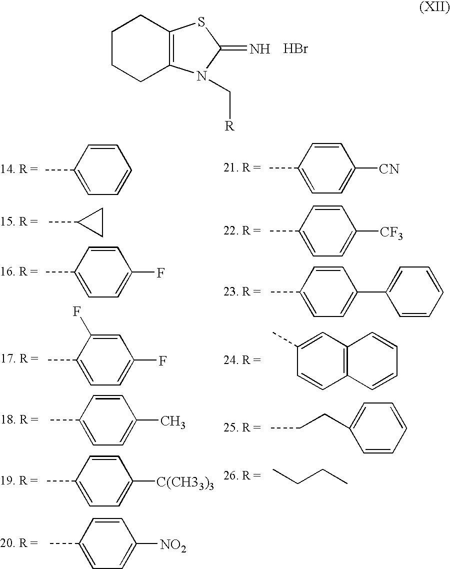

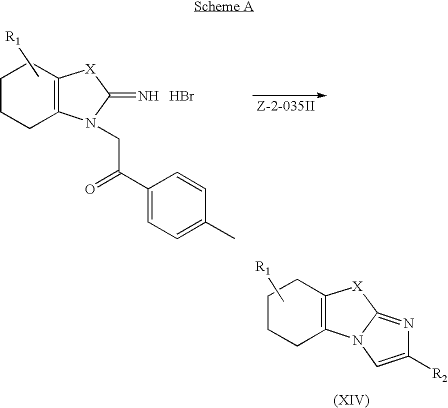

Z-2-035II: 2-(4′-methylphenyl)-5,6,7,8-tetrahydroimidazo[2,1-b]benzothiazole (29), mp 187° C. (MeOH/EtOAc) (lit 185° C.); ¹H NMR (DMSO-d₆) δ 7.71 (d, J=8.1 Hz, 2H), 7.50 (s, 1H), 7.19 (d, J=8.1 Hz, 2H), 2.70-2.63 (m, 4H), 2.35 (s, 3H), 1.95-1.93 (m, 4H).

| Compound | R₁ | X |
|---|---|---|
| 29 | H | S |
| | CH₃ | O |

wherein R₂ is C₁₋₈ alkyl or aryl substituted or unsubstituted

### Example 3

A mixture of 2,3,4,5,6,7-hexahydrobenzothiazolin-2-one (0.1 mol) and sodium hydride (0.1 mol) in toluene (20 ml) was refluxed for half an hour. 2-bromo-4′-methylacetophenone (0.1 mol) was added and the reaction mixture was refluxed for an additional four hours. The precipitated sodium bromide was filtered off and the toluene was removed under vacuum. The crude product was purified to give 3-p-methylphenacyl-2,3,4,5,6,7-hexahydrobenzothiazolin-2-one (XIII) in the yield of 48%. ¹HNMR (CDCl₃): 7.88 (d, J=8.0 Hz, 2H), 7.28 (d, J=8.0 Hz, 2H), 5.04 (s, 2H), 2.42 (br, 5H), 2.19-2.17 (m, 2H), 1.82-1.78 (m, 4H).

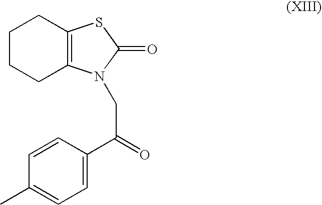

### Example 4

### Neuroprotective Activity of PFTα, its Precursor, and Analogues in PC12 cells

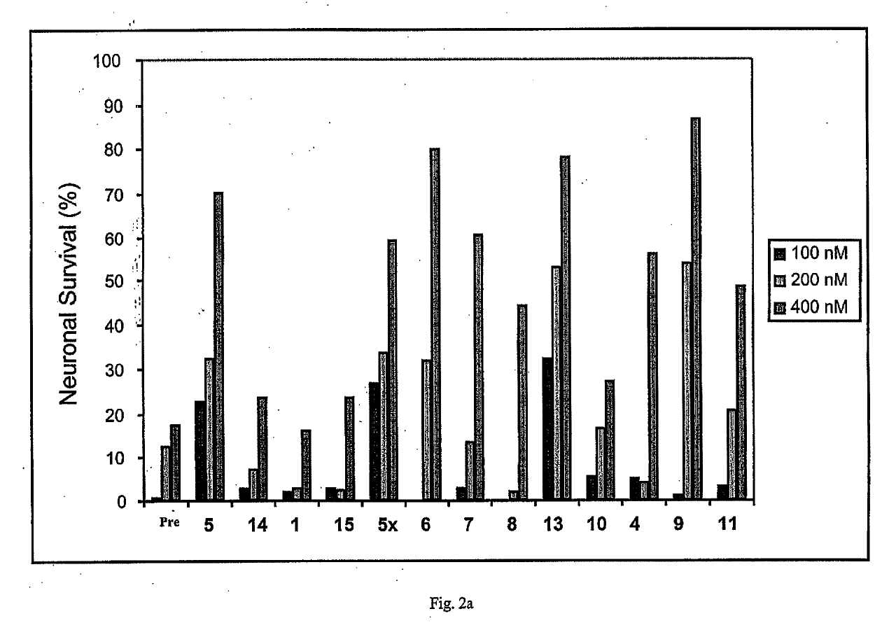

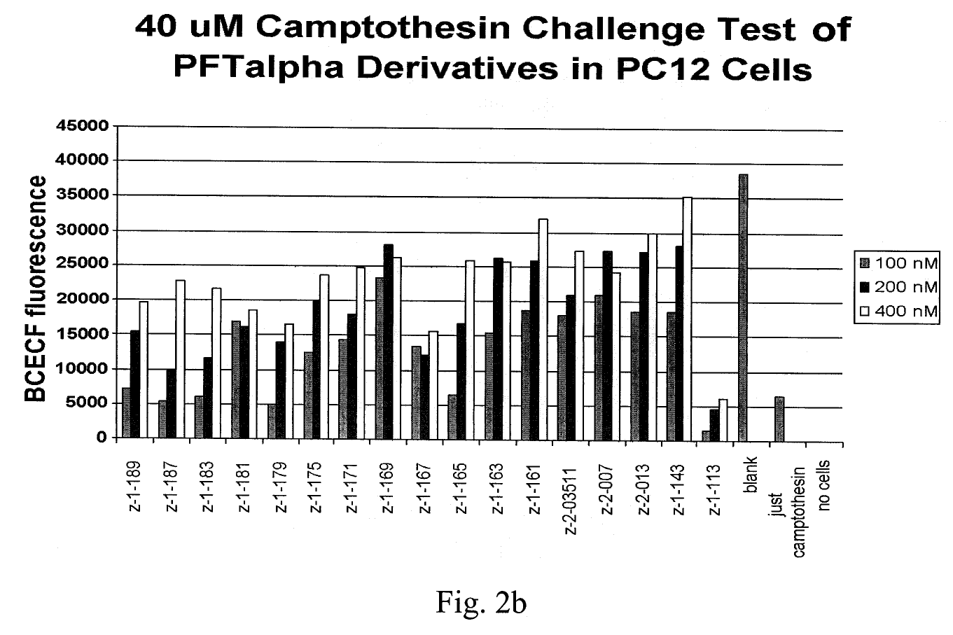

The 2-amino-4,5,6,7-tetrahydrobenzothiazole hydrogen iodide, 1-(4-methylphenyl)-2-(4,5,6,7-tetrahydro-2-imino-3(2H)-benzothiazolyl)-ethanone hydrobromide, and analogues were tested for their ability to protect PC12 cells against the toxic agent, camptothecin. See FIGS. 2a and 2b. PC12 cells were cultured in RPMI1640 media containing 10% horse serum and 5% fetal calf serum (37° C., pH 7.4) until confluent. Thereafter, 2.2×10⁴ cells per mL were added to each trough of a 96 well plate (approximately 6,270 cells per well 285 μL) and were left to grow for 12 h. The media then was removed and was replaced by media with and without the compounds to be tested, including precursor, PFTα, and numerous analogues (concentration 100-400 nM, prepared in DMSO to a final dilution of 0.4%, 8 wells per concentration). Following 6 h of incubation, camptothecin (40 μM) was added to 4 of each 8 wells, and the cells were incubated for a further 24 h. Cells then were gently washed twice with phosphate buffered saline (PBS: 0.1 M, 37° C., pH 7.4) and were incubated in PBS containing the live-cell indicator dye, 2′,7′-bis(2-carboxyethey)-5-6-carboxyfluorescein AM ester (BCECF AM: 5 μM) (Molecular Probes, Calif.), for 45 min in minimal light. This fluorescent dye was taken up and retained by live cells. Finally, the cells were washed twice in PBS and their fluorescence then was quantified (excitation: 490 nm, emission: 535 mm, Perkin Elmer HTS 7000). See FIG. 2A.

PC12 cell survival was also assessed. The results are shown in FIG. 2B. One hundred percent cell survival was determined from cells incubated in the absence of both any compound and camptothecin. One hundred percent cellular death was determined from cells incubated in the absence of any compound and presence of camptothecin, which in prior studies was found to occur at a minimum concentration of 40 μM. Compound-induced toxicity was assessed by comparing cells treated with compound at various concentrations in the absence of camptothecin with cells incubated in the absence of both compound and camptothecin. In the presence of PFTα, neuronal survival of PC12 cells was increased up to 70% as compared to the PC12 cells treated with the DNA damaging agent, camptothecin, in the absence of PFTα. The precursor, 2-amino-4,5,6,7-tetrahydrobenzothiazole hydrogen iodide, showed low activity under the same conditions. No compound was found to induce PC12 cell toxicity at the maximal concentration assessed (400 nM). Compound-induced cellular protection was assessed by comparing cells incubated with camptothecin with cells incubated with camptothecin in the presence of compound, in a concentration-dependent manner. Finally, the natural fluorescence of each compound was quantified in the absence of cells and BCECF AM, and was found to be negligible. A 50% protective concentration (PC₅₀), commensurate with an IC₅₀ (concentration required to inhibit 50% of p53 activity), was calculated by transforming the data into a logit format (logit=ln(% protection/100%-% protection)), and calculating the value from a correlation between a plot of the log concentration of compound versus logit activity.

In summary, compounds 5x, 6-7, 9, 13, proved to be highly potent in protecting PC12 cells. The concentration of each analogue required to protect 50% of cells (determined as a PC₅₀ value) is shown in Table 1.

TABLE 1
Concentration of compounds required to induce 50% protection
(PC₅₀) of PC₁₂ cells from camptothecin-induced cell-death.

| Compound | | PC₅₀ nM |
|---|---|---|
| (precursor) | | 5754 |
| 5 | (PFTα) | 252 |
| 14 | (Z-119) | 741 |
| 1 | (Z-110) | 571 |
| 15 | (Z-113) | 728 |
| 5x | (Z-117) | 306 |
| 6 | (Z-133) | 182 |
| 7 | (Z-138) | 348 |
| 8 | (Z-139) | 418 |
| 13 | (Z-143) | 169 |
| 10 | (Z-145) | 767 |
| 4 | (Z-135) | 379 |
| 9 | (Z-141) | 187 |
| 11 | (Z-153) | 410 |
| 16 | (Z-1-181) | 154 |
| 17 | (Z-1-163) | 93 |
| 18 | (Z-1-179) | 1000 |
| 19 | (Z-1-165) | 315 |
| 20 | (Z-1-161) | 144 |
| 21 | (Z-1-183) | 418 |
| 22 | (Z-1-175) | 327 |
| 24 | (Z-1-171) | 318 |
| 25 | (Z-1-167) | 1100 |
| 26 | (Z-1-187) | 395 |
| 2 | (Z-1-189) | 595 |
| 30 | (Z-1-169) | 133 |
| 27 | (Z-2-007) | 69 |
| 28 | (Z-2-013) | 143 |
| 29 | (Z-2-035II) | 214 |

In summary, numerous of the analogues (e.g. 5x, 6-7, 9-13, etc.) prove to be highly potent.

### Example 5

### Neuroprotective Activity of PFTα and its Precursor in Rat Hippocampal Neurons In Vitro

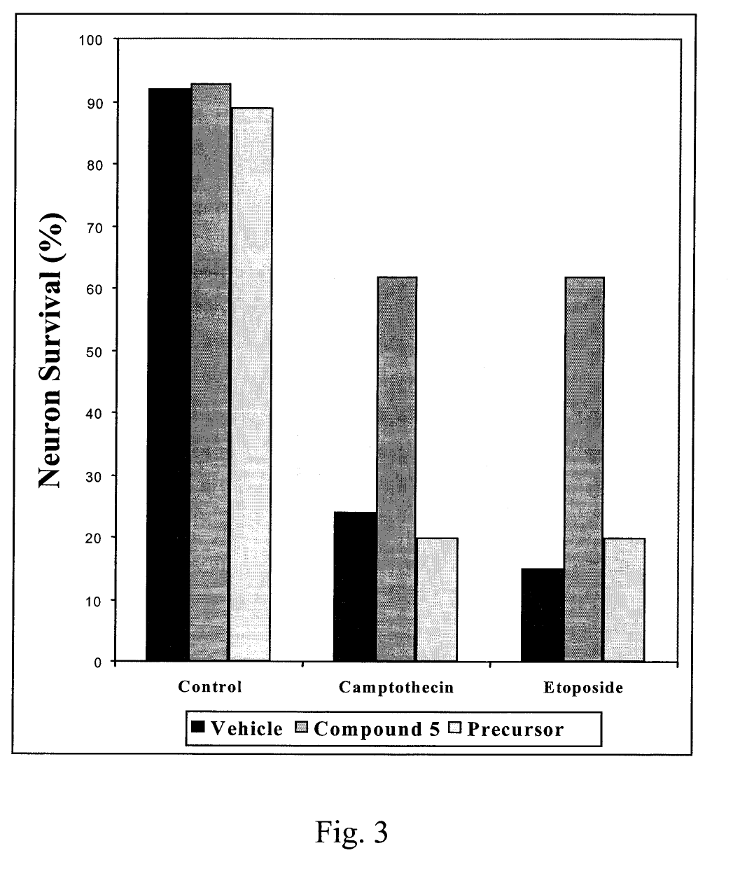

2-amino-4,5,6,7-tetrahydrobenzothiazole hydrogen and 1-(4-methylphenyl)-2-(4,5,6,7-tetrahydro-2-imino-3(2H)-benzothiazolyl)-ethanone hydrobromide and their analogues were assessed for their ability to protect rat hippocampal cells in culture from camptothecin (5 μM) or etoposide (5 μM). See FIGS. 3-5.

Dissociated hippocampal cell cultures were established from embryonic day 18 Sprague-Dawley rats (Harlan, Inc.) as described previously. See Culmsee et al. (2001) J. Neurochem. 77:220-28. Cells were grown in polyethyleneimine-coated plastic dishes or 22 mm glass coverslips, and incubated in Neurobasal medium containing B-27 supplements, 2 mM L-glutamine, 25 mg/ml gentamycin, 1 mM Hepes (Gibco BRL) and 0.001% gentamicin sulfate. All experiments were performed using 9-10 day-old cultures. Camptothecin and etoposide (Sigma) were prepared as 500× stocks in DMSO. Neuron survival was quantified by established methods. See Mattson et al. (1993) Neuron 10:243-54. Briefly, viable neurons in premarked fields (10× objective) were counted before experimental treatment and at specified time points thereafter. Neurons with intact neurites of uniform diameter and soma with a smooth round appearance were considered viable. In contrast neurons with fragmented neurites and vacuolated soma were considered non-viable.

The results showed that PFTα (5) possessed potent activity to protect the primary hippocampal cells. At concentrations up to 400 nM, neither PFTα (5) nor its precursor were toxic when administered alone to cells. However, both camptothecin (5 μM) and etoposide (2.5 μM), known to induce neuronal apoptosis by a mechanism involving DNA damage and p53 induction, killed approximately 85% of neurons. Pretreatment of cells with PFTα (5), but not its precursor, resulted in improved cell survival compared to vehicle controls.

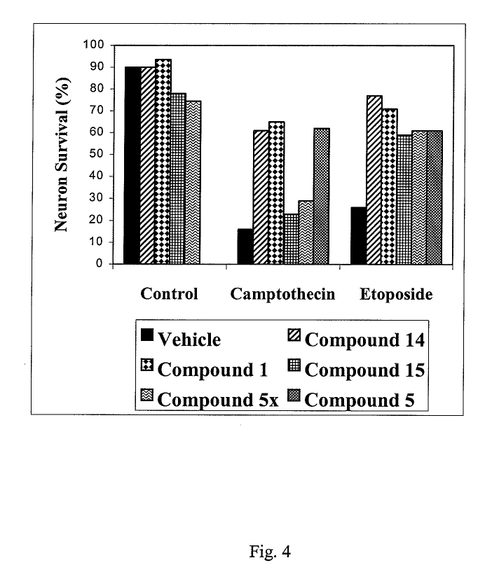

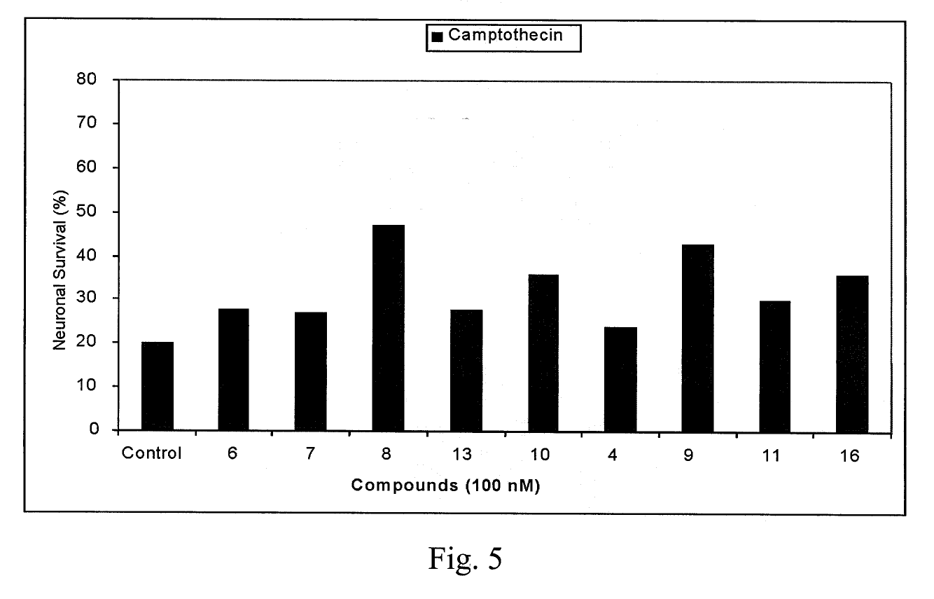

The neuroprotective effect of the analogues was also tested. At concentrations of 100 nM none of compounds 14, 1, 15, 5x, 6-11, 13, 16 were toxic when only the analogues were put in the cell. When the hippocampal neurons were pretreated with 100 nM of analogue and then exposed to camptothecin, the neuronal survival for compounds 14, 1, 15, 5x and 5 was 61%, 65%, 23%, 29% and 62% respectively. See FIGS. 4 and 5. Compounds 14 and 1 had about the same neuroprotective activity as PFTα and exhibited significant neuroprotection against the DNA-damage agent camptothecin. Compound 5x, a 5-methyl substituted derivative of compound 5, and compound 15 could not protect neurons effectively against camptothecin (a topisomerase I inhibitor). When these analogues 14, 1, 15, 5x, 6-11, 13, 16 were tested their ability to prevent neuron against etoposide, compound 14 and compound 1 were more active than compound PFTα, and compound 15 and compound 5x have activity comparable to PFTα. Compounds 14, 1, 15, 5x, and 5 had activity against the topisomerase II inhibitor, etopside.

Compound 14 and compound 1 had neuroprotection against death induced by both camptothecin and etoposide. When these two compounds were selected for further study, they were also shown to suppress caspase activation and protect cultured hippocampal neurons against death induced by glutamate. Neuron survival was increased 133% and 39% respectively when neurons were pretreated with compound 14 or 1.

Thus, compounds 14, 1, 15, 5x, 8 and 9 proved to be highly potency in protecting primary hippocampal cells from apoptosis induced cell death. The N-substituent group of compound 5 proved to be essential for biological action. Modification of N-substituent to omit the carbonyl group, compound 14, resulted in a compound that retained biological activity in the protection of hippocampal cells from both etopside and camptothecin. 4-Methyl substitution in the phenyl position of the N-substituent proved to be non essential for biological action, with compound 13 proving to be highly active in PC12 cells only. Indeed, substitution of a simple alkyl, compound 1, for the aryl group in the N-substituent was well tolerated to provide high activity in hippocampal cells. Substitution of electron donating (e.g., OCH₃ as in compound 6) and withdrawing (e.g., Cl as in compound 9) groups, particularly in the 4′-position of the phenyl group of the N-substituent, were well tolerated. Methyl substitution in position 5, as in compound 5x, was well tolerated and provided an agent active in all assays.

### Example 6

### Neuroprotective Activity of PFTA (2) and its Precursor in an In Vivo Stroke Model

Surgery was performed in anesthetized (xylazine 5 mg/kg, chloral hydrate 350 mg/kg) mice (3 month old C57B1/6) in accordance with a protocol approved by the NIH Animal Care and Use Committee. The focal ischemia/reperfusion model has been described previously. See Bruce et al. (1996) Nat. Med. 2:788-94. This method involves occluding the middle cerebral artery for 1 h with a nylon thread, and then removing it to allow reperfusion. During the procedure, mice were maintained at 37° C. and blood gases, flow and pressure were monitored. At 24 h after ischemia, mice were anesthetized, decapitated, and their brains were cut into 2 mm coronal sections, which then were stained with triphenyltetrazolium for 30 min at 37° C. Images of the stained brains were captured by digital camera for quantitative analysis of infarct area and volume.

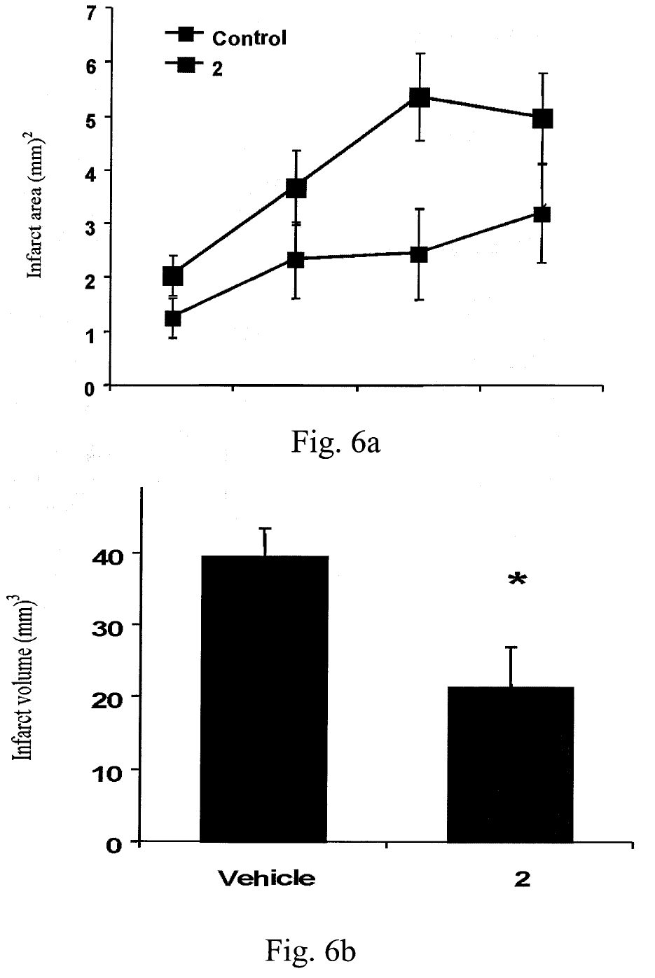

PFTα (5) (2 mg/kg, i.p. in 0.2% DMSO) administered 30 min prior to the initiation of cerebral ischemia exhibited neuroprotective activity. FIG. 6A shows the infarct area at four different brain levels, with a reduction in the infarct area following PFTα. The infarct volume for vehicle was 40 mm³. When mice were treated with PFTα, infarct volume was 20 mm³, a highly significant 50% decrease. See FIG. 6B. Thus, after systemic administration, PFTα entered and acted in the brain.

### Example 7

### Neuroprotective Activity of PFTA and its Analogues in an In Vivo Model of Parkinson's Disease

The ability of PFTα (5) and compounds 5x (Z-1-117) and 13 (Z-1-143) to protect dopaminergic neurons against MPTP toxicity was tested. MPTP (20 mg/kg) was administered intraperitoneally to 2 month-old male C57BL/6 mice by four injections at two hour intervals to induce dopaminergic cell death. Animals were pretreated with either PFTα (2 mg/kg), compound 5x (2 mg/kg), compound 13 (2 mg/kg), or vehicle (0.2% DMSO) by intraperritoneal injection 30 min. prior to the first MPTP injection and 30 min. after the last MPTP injection.

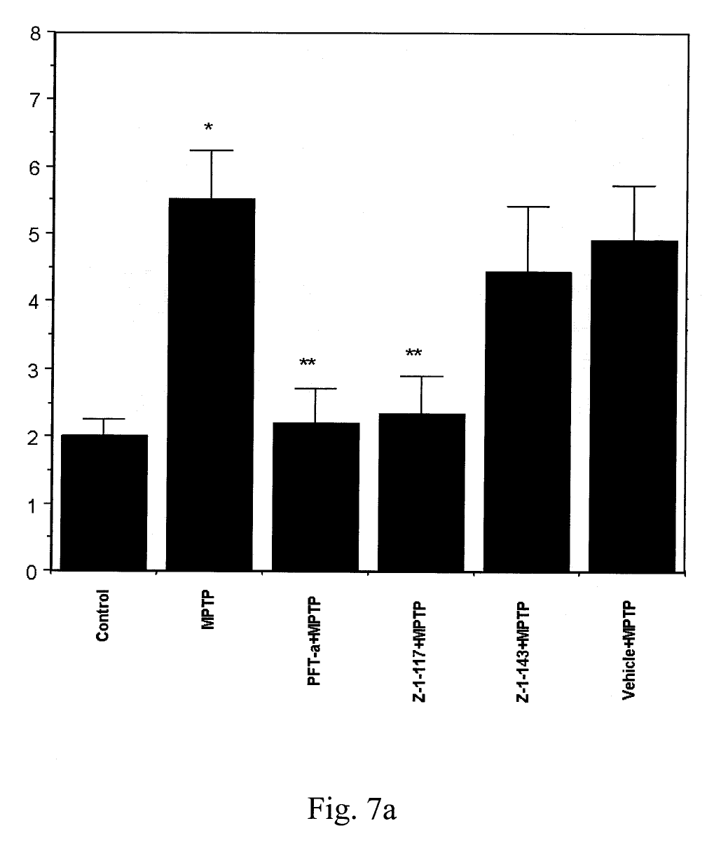

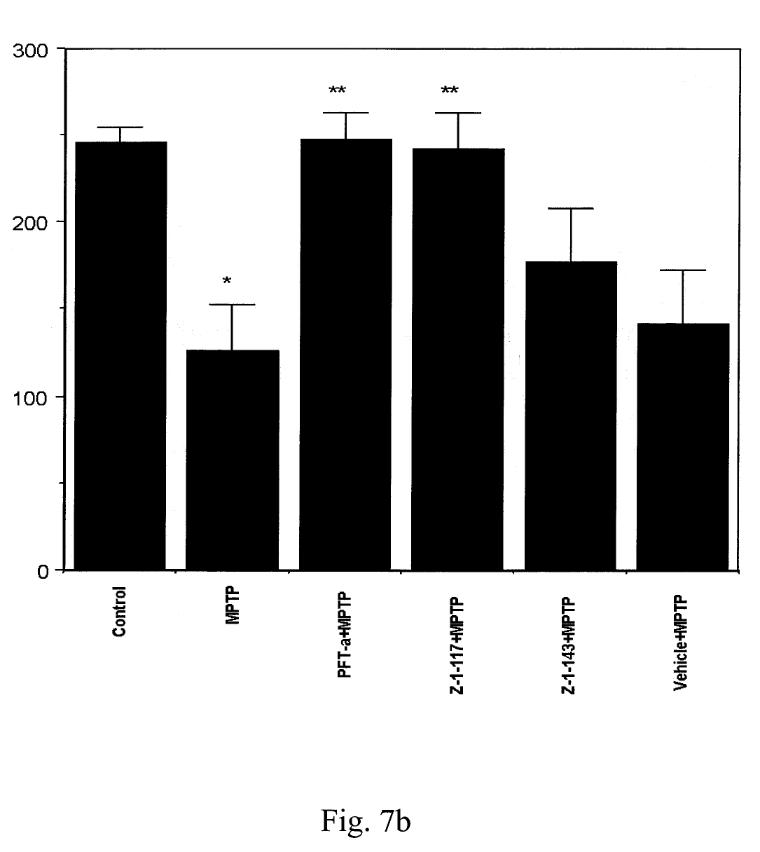

At seven days, behavioral tests were performed using the rotarod test equipment. The number of falls and running times were measured. See FIG. 7. Treatment with PFT-α and compound 5x resulted in a statistically significant decease in the number of falls caused by MPTP treatment. See FIG. 7A. Also, PFT-α and compound 5x resulted in a statistically significant increase in the running time, which is markedly reduced by MPTP treatment as compared to control. See FIG. 7B.

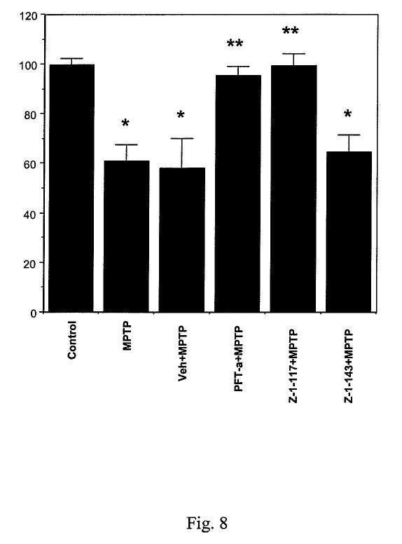

Levels of tyrosine hydroxylase (TH), the enzyme that catalyzes the rate-limiting step in dopamine synthesis, also were assessed in the striatum using Western blot analysis. See FIG. 8. Densitometric analysis shows that PFT-α and compound 5x reverse the MPTP reduction in TH levels.

In a separate experiment, levels of striatal dopamine, dopamine metabolites, serotonin, and serotonin metabolites were measured using HPLC analysis. Specifically, mice were euthanatized, and striatal tissue samples were removed and levels of dopamine, DOPAC, HVA, 5-HT, 5-HLAA were quantified. The data are shown in Table 2. PFT-α and compound (5x) resulted in a statistically significant increase in levels of dopamine, DOPAC, and HVA as compared to the MPTP-induced reduction over control. There was no significant difference in levels of serotonin or serotonin metabolites.

TABLE 2

| | Dopamine | DOPAC | HVA | 5-HT | 5-HIAA |
|---|---|---|---|---|---|
| Control | 6178 ± 799 | 337 ± 42 | 618 ± 53 | 486 ± 33 | 298 ± 29 |
| MPTP | 405 ± 56 | undetectable | 124 ± 53 | 441 ± 22 | 239 ± 24 |
| Vehicle + | 404 ± 93 | undetectable | 125 ± 37 | 406 ± 17 | 243 ± 18 |
| MPTP | | | | | |
| PFT-α (5) + | 3314 ± 670** | 170 ± 25** | 328 ± 35** | 419 ± 20 | 245 ± 33 |
| MPTP | | | | | |
| Z-1-117 (5x) + | 1833 ± 256* | 102 ± 11** | 302 ± 20** | 424 ± 12 | 246 ± 15 |
| MPTP | | | | | |
| Z-1-143 (13) + | 440 ± 122 | 10 ± 10 | 91 ± 58 | 429 ± 42 | 223 ± 45 |
| MPTP | | | | | |

Values (pg/mg tissue weight) are the mean ± SE of determinations made in 4-6 mice/group.
*P < 0.05,
**P < 0.01 compared to the value of MPTP group. ANOVA with Scheffe post-hoc tests.

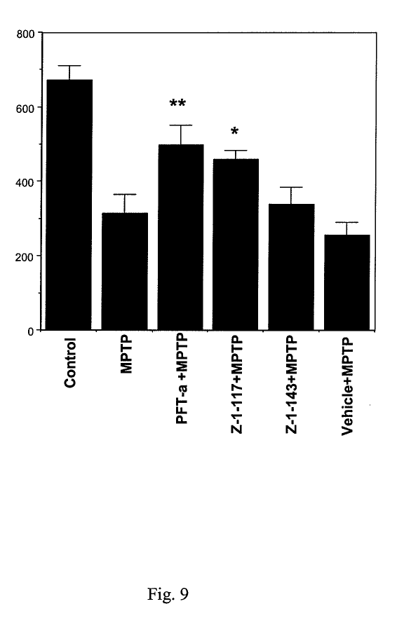

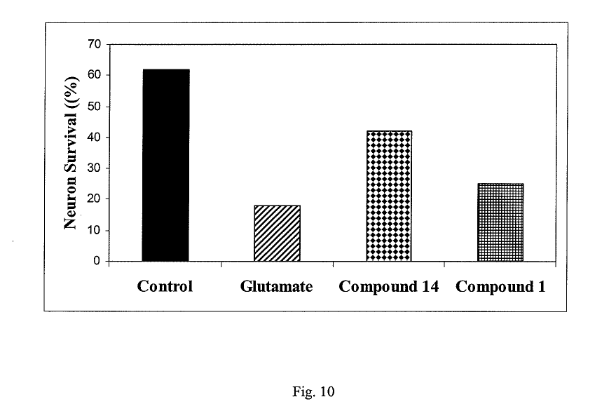

In yet another experiment, the number of TH-immunostained cells in the substantia nigra was assessed in four animals seven days after treatment. Mice were euthanized and perfused by 4% paraformadehyde transcardially. Brain sections were cut and immunostained with an antibody against TH. PFT-α and compound 5x attenuated MPTP-induced loss of substantia nigra dopaminergic neurons. See FIG. 9.

### Example 8

### Neuroprotective Activity of PFT Analogues to an Excitotoxic Insult

Primary hippocampal neurons were prepared and cultured as described above. Cultures were pretreated with compounds 14 and 1 (0.04 μM) 1 h before exposure to glutamate (20 μM) in Locke's medium. The percentage of neuronal survival 24 h after the onset of insult is given as mean values ±SD. Glutamate resulted in a marked decrease in neuronal survival. The glutamate-induced decrease was at least partially reversed by compound 14, thus demonstrating a neuroprotective effect. See FIG. 10.

### Example 9

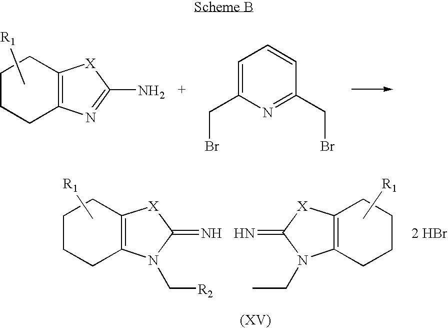

### Z-1-169: 2,6-Bis[(4,5,6,7-tetrahydro-2-imino-3(2H)-benzothiazolyl)methyl]pyridine dihydrobromide (30)

This was prepared by a similar procedure from 2-amino-4,5,6,7-tetrahydrobenzothiazole (109 mg, 0.7 mmol) and 2,6-Bis(bromomethyl)pyridine (95 mg, 0.35 mmol); yield (61%); ¹H NMR (DMSO-d₆) δ 9.29 (s, 2H), 7.96 (t, J=7.7 Hz, 1H), 7.47 (d, J=7.7 Hz, 2H), 5.33 (s, 4H), 2.56 (m, 4H), 2.12 (m, 4H), 1.68 (m, 8H); Anal. (C₂₁H₂₇Br₂N₅S₂) C, H, N.

In formula (XV) above, R₁ is H, C₁₋₈ alkyl or aryl, substituted or unsubstituted; R₂ is any aromatic ring, substituted or unsubstituted and X is S or O.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A tetrahydrobenzothiazole analogue having the formula (I): 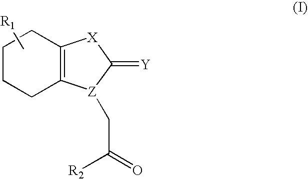
or a pharmaceutically acceptable salt or ester thereof, wherein:
X is O;
Y is NH, O, NR₂ or S; 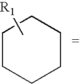
is a mono-substituted, di-substituted, tri-substituted, or quad-substituted 6-member ring that is saturated or unsaturated, wherein the R₁ substituents are independently chosen from H, CN, NO2, S, N, O, OH, COOH, halogen, and R₂, but not methyl; and
Z is N;
R₂ is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy, wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

2. A pharmaceutical composition comprised of a compound of claim 1 in combination with a pharmaceutically acceptable carrier.

3. A tetrahydrobenzothiazole analogue having the formula (II): 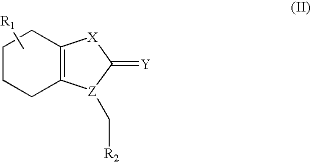
or a pharmaceutically acceptable salt or ester thereof, wherein:
X is O;
Y is NH, O, NR₂ or S; 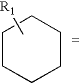
is a mono-substituted, di-substituted, tri-substituted, or quad-substituted 6-member ring that is saturated or unsaturated, wherein the R₁ substituents are independently chosen for H, CN, NO2, S, N, O, OH, COOH, halogen, and R₂, but not methyl; and
Z is N;
Ris selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

4. A pharmaceutical composition comprised of a compound of claim 3 in combination with a pharmaceutically acceptable carrier.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A tetrahydrobenzooxyzole analogue having the formula (IX): 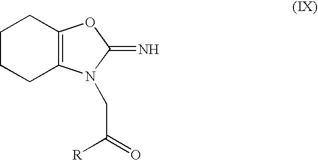
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

24. An analogue of claim 23, wherein R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

25. A pharmaceutical composition comprised of a compound in accordance with claim 23 in combination with a pharmaceutically acceptable carrier.

26. A tetrahydrobenzooxyzole analogue having the formula (X): 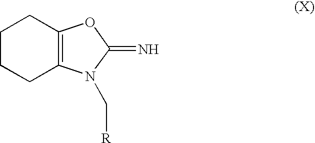
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

27. An analogue of claim 26, wherein R is methyl, benzyl, cyclopropylmethyl, fluorobenzyl, difluorobenzyl, chlorobenzyl, dichlorobenzyl, methylbenzyl, t-butylbenzyl, nitrobenzyl, cyanobenzyl, trifluoromethylbenzyl, phenylbenzyl, menaphthyl, phenylethyl, or butyl.

28. A pharmaceutical composition comprised of a compound in accordance with claim 26 in combination with a pharmaceutically acceptable carrier.

29. A method of reducing or delaying apoptosis in a population of cells, comprising contacting the population of cells with a tetrahydrobenzothiazole analogue, thereby reducing or delaying apoptosis in the population of cells.

30. (canceled)

31. The method of claim 29, wherein the tetrahydrobenzothiazole analogue comprises one or more analogues of formula (I): 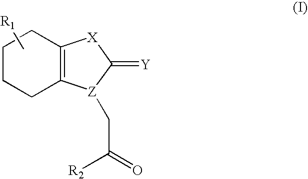
or a pharmaceutically acceptable salt or ester thereof, wherein:
X is O;
Y is NH, O, NR₂ or S; 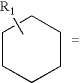
is a mono-substituted, di-substituted, tri-substituted, or quad-substituted 6-member ring that is saturated or unsaturated, wherein the R₁ substituents are independently chosen from H, CN, NO2, S, N, O, OH, COOH, halogen, and R₂ but not methyl; and
Z is N;
R₂ is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy, wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

32. The method of claim 29, wherein the tetrahydrobenzothiazole analogue comprises one or more analogues of formula (II): 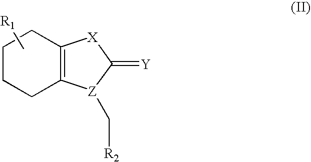
or a pharmaceutically acceptable salt or ester thereof, wherein:
X is O;
Y is NH, O, NR₂ or S; 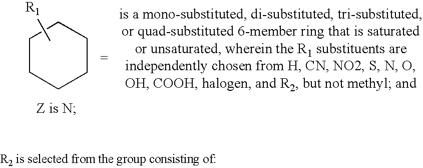
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

33. (canceled)

34. (canceled)

35. (canceled)

36. (canceled)

37. (canceled)

38. (canceled)

39. The method of claim 29, wherein the tetrahydrobenzothiazole analogue comprises one or more analogues of formula (IX): 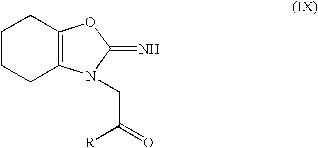
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

40. The method of claim 29, wherein the tetrahydrobenzothiazole analogue comprises one or more analogues of formula (X): 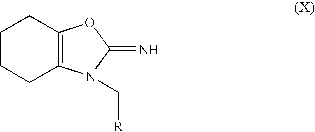
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

41. The method of claim 29, wherein the contacting step is in vivo.

42. The method of claim 29, wherein the contacting step is in vitro.

43. The method of claim 29, wherein the cells are neural cells.

44. The method of claim 43, wherein the neural cells are neuronal cells.

45. The method of claim 29, wherein the cells are cardiac cells.

46. The method of claim 45, wherein the cells are cardiomyocytes.

47. The method of claim 29, wherein the cells are muscle cells.

48. The method of claim 47, wherein the muscle cells are skeletal muscle cells.

49. The method of claim 29, wherein the cells are pancreatic islet cells.

50. The method of claim 29, wherein the apoptosis is induced by a toxin.

51. The method of claim 29, wherein the apoptosis is induced by an environmental factor.

52. The method of claim 29, wherein the apoptosis is induced by a degenerative condition.

53. The method of claim 29, wherein the apoptosis is induced by a severe seizure disorder.

54. The method of claim 29, wherein the apoptosis is induced by a genetic disease.

55. The method of claim 50, wherein the toxin is selected from the group consisting of a neurotoxic form of amyloid β-peptide, camptothecin, glutamate, etoposide, anti-cancer drugs, vinca alkaloids, 3-nitrognognonic acid, MPTP, domoic acid, and kainic acid.

56. The method of claim 29, wherein the apoptosis is induced by ischemia.

57. The method of claim 52, wherein the ischemia is induced by a stroke.

58. The method of claim 42, wherein the ischemia is induced by a myocardial infarction.

59. The method of claim 29, wherein the apoptosis is induced by trauma.

60. The method of claim 29, wherein the apoptosis is induced by a genetic defect.

61. A method of treating a subject with a degenerative condition or of reducing one or more symptoms of a degenerative condition in a subject, comprising administering to the subject a therapeutically effective amount of a tetrahydrobenzothiazole analogue.

62. (canceled)

63. The method of claim 61, wherein the tetrahydrobenzothiazole analogue comprises one or more analogues of formula (I): 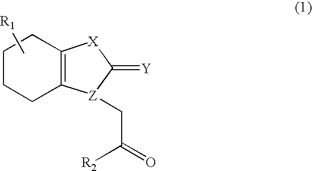
or a pharmaceutically acceptable salt or ester thereof, wherein:
X is O S;
Y is NH, O, NR₂ or S;
Z is N; 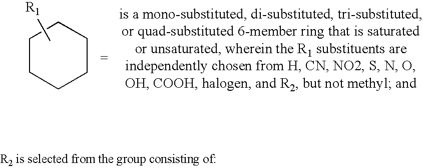
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy, wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

64. The method of claim 61, wherein the tetrahydrobenzothiazole analogue comprises one or more analogues of formula (II): 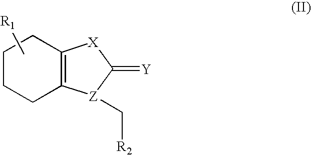
or a pharmaceutically acceptable salt or ester thereof, wherein:
X is O;
Y is NH, O, NR₂ or S;
Z is N; 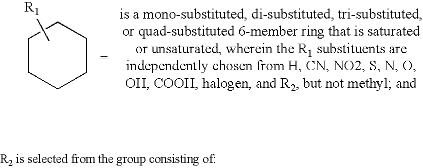
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

65. (canceled)

66. (canceled)

67. (canceled)

68. (canceled)

69. (canceled)

70. (canceled)

71. The method of claim 61, wherein the tetrahydrobenzothiazole analogue comprises one or more analogues of formula (IX): 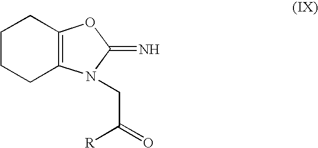
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

72. The method of claim 61, wherein the tetrahydrobenzothiazole analogue comprises one or more analogues of formula (X): 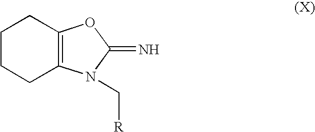
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

73. The method of claim 61, wherein the degenerative condition is a neurodegenerative condition.

74. The method of claim 73, wherein the neurodegenerative condition is selected from the group consisting of Alzheimer's disease, Parkinson's disease, Huntington's disease, amyotrophic lateral sclerosis, stroke, multiple sclerosis, brain injury, spinal cord injury, and peripheral neuropathy.

75. The method of claim 61, wherein the degenerative condition is a degenerative cardiomyopathy.

76. The method of claim 61, wherein the degenerative condition is a degenerative myopathy.

77. The method of claim 61, wherein the degenerative condition is diabetes.

78. The method of claim 61, wherein the subject is a human.

79. A method of treating a subject after an ischemic event to reduce ischemia-induced apoptosis, comprising administering to the subject a therapeutically effective amount of a tetrahydrobenzothiazole analogue, thereby reducing apoptosis.

80. (canceled)

81. The method of claim 79, wherein the tetrahydrobenzothiazole analogue comprises one or more analogues of formula (I): 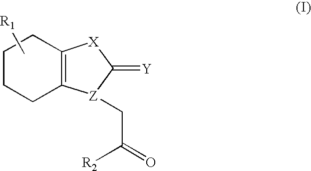
or a pharmaceutically acceptable salt or ester thereof, wherein:
X is O;
Y is NH, O, NR₂ or S; 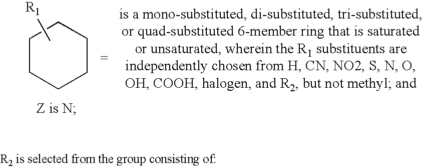
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one carbon atom or from 3 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₂-C₂₀ alkyl, C₁-C₂₀ alkenyl, C₁-C₂₀ alkynyl, and C₁-C₂₀ alkoxy, wherein the halogen is not in the para position;
(d) bisaryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

82. The method of claim 79, wherein the tetrahydrobenzothiazole analogue comprises one or more analogues of formula (II): 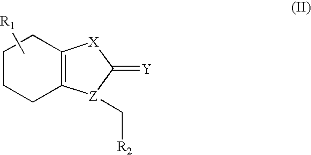
or a pharmaceutically acceptable salt or ester thereof, wherein:
X is O;
Y is NH, O, NR₂ or S; 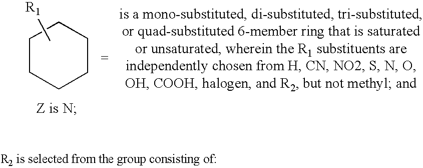
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of one to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

83. (canceled)

84. (canceled)

85. (canceled)

86. (canceled)

87. (canceled)

88. (canceled)

89. The method of claim 79, wherein the tetrahydrobenzothiazole analogue comprises one or more analogues of formula (IX): 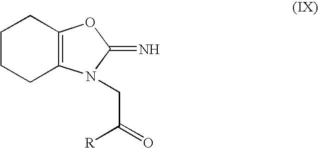
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

90. The method of claim 79, wherein the tetrahydrobenzothiazole analogue comprises one or more analogues of formula (X): 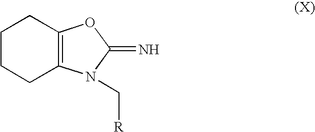
or a pharmaceutically acceptable salt or ester thereof, wherein:
R is selected from the group consisting of:
(a) alkyl, or alkenyl, or alkynyl, each of which are straight chain, branched chain or cyclic, having a carbon chain length of from 1 to 20 carbon atoms, unsubstituted or substituted with at least one member selected from the group consisting of CN, NO₂, S, N, OH, COOH, and halogen;
(b) alkoxy which is straight chain, branched chain or cyclic, having a carbon chain length of from 1 to 20 carbon atoms, unsubstituted or substituted with at least one member selected from the group consisting of CN, NO₂, S, N, O, OH, COOH, halogen, C₁-C₂₀ alkyl, C₁-C₂₀ alkenyl, and C₁-C₂₀ alkynyl;
(c) aryl which is substituted with at least one member selected from the group consisting of CN, NO₂, S, N, O, OH, COOH, halogen, C₁-C₂₀ alkyl, C₁-C₂₀ alkenyl, C₁-C₂₀ alkynyl, and C₁-C₂₀ alkoxy;
(d) bisaryl which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl; and
(e) condensed aromatic which is unsubstituted or substituted with at least one member selected from the group consisting of CN, NO₂, S, N, O, OH, COOH, halogen, C₁-C₂₀ alkyl, C₁-C₂₀ alkenyl, C₁-C₂₀ alkynyl, C₁-C₂₀ alkoxy, and aryl.

91. The method of claim 79, wherein the ischemic event is a stroke.

92. The method of claim 79, wherein the ischemic event is a myocardial infarction.

93. The method of claim 79, wherein the subject is a human.

94. (canceled)

95. (canceled)

96. (canceled)

97. (canceled)

98. (canceled)

99. (canceled)

100. (canceled)
